(19) United States
(12) Patent Application Publication
(10) Pub. No.: US 2022/0249669 A1
(43) Pub. Date: Aug. 11, 2022

(54) ULTRASOUND-TRIGGERED LIPOSOME PAYLOAD RELEASE

(71) Applicants: IMPERIAL COLLEGE INNOVATIONS LTD., London (GB); THE CHANCELLOR, MASTERS AND SCHOLARS OF THE UNIVERSITY OF OXFORD, Oxfordshire (GB)

(72) Inventors: Valeria NELE, LONDON (GB); James P. ARMSTRONG, LONDON (GB); Molly M. STEVENS, LONDON (GB); Carolyn SCHUTT IBSEN, LONDON (GB); Michael D. GRAY, OXFORD (GB); Constantin C. COUSSIOS, OXFORD (GB)

(21) Appl. No.: 17/631,766

(22) PCT Filed: Jul. 31, 2021
(86) PCT No.: PCT/GB2020/051847

§ 371 Date: Jan. 31, 2022

(30) Foreign Application Priority Data
Aug. 1, 2019 (GR) 20190100331
Aug. 6, 2019 (GB) 1911235.8

(51) Int. Cl. A61K 41/00; A61K 49/22; A61K 47/42; A61K 47/02; A61K 47/10; A61K 47/36; A61K 47/18; A61K 47/24; A61K 9/127; C12N 9/10

(52) U.S. Cl. CPC A61K 41/0047; A61K 49/227; A61K 41/0028; A61K 47/42; A61K 47/02; A61K 47/10; C12Y 203/02013; A61K 47/183; A61K 47/24; A61K 9/1271; A61K 49/223; A61K 49/226; C12N 9/1044; A61K 47/36

(57) ABSTRACT

Described herein are processes and compositions for ultrasound-triggered liposome payload release, including a process for gelation and a process for enzyme catalysis.

### FIELD OF INVENTION

The invention relates to ultrasound-triggered liposome payload release and its use in, for example, the formation of hydrogels through ultrasound-triggered gelation.

### BACKGROUND

Hydrogels are hydrated, three-dimensional polymeric networks that are widely used for applications in tissue engineering, drug delivery, soft robotics and bioelectronics. The base materials encompass a broad range of hydrophilic homopolymers, copolymers or macromers, which can be natural (e.g. collagen, alginate, fibrin), fully synthetic (e.g. poly(ethylene glycol), poly(vinyl alcohol), poly(acrylic acid)) or semi-synthetic (e.g. methacrylate-, tetrazine-, norbornene-modified biopolymers). Hydrogels are formed through sol-gel transitions mediated by the formation of various noncovalent or covalent bonds. For instance, many hydrogels are crosslinked by ions, small molecules or peptides, which form chemical bonds that bridge adjacent polymer chains. However, the need for a second component to be added to the system presents challenges for many applications, in particular in vivo gelation.

Hydrogelation can also be initiated by changing environmental conditions, such as temperature or pH. These stimuli can be used to directly alter the chemical environment of the material through changes in noncovalent interactions, or alternatively trigger the release of chemical factors to initiate gelation. This strategy is used for injectable formulations that are designed to gel under physiological conditions, however, these systems are typically limited by poor spatiotemporal control.

One method that can achieve high spatiotemporal precision is the use of ultraviolet or blue light irradiation to photocrosslink synthetic or semi-synthetic hydrogels. Yet, photo-crosslinking applications can be hindered by the common need for radical photoinitiators, as well as the limited tissue penetration of light at these wavelengths.

In 2008, Park et al. (Soft Matter 2008, 4, 1995), the entire contents of which are herein incorporated by reference, used ultrasound to generate radicals that could initiate the formation of 2′-deoxyadenosine-based hydrogels. However, the gelation was induced by the formation of hydroxyl radicals, which may be detrimental for biomedical applications.

Therefore, it is desirable to provide an alternative means for initiating hydrogelation and, more generally, enzyme catalysis.

### SUMMARY OF THE INVENTION

In a first aspect, the invention provides a process for gelation (for example, hydrogelation), wherein the process comprises the steps of:
a) providing a mixture comprising a liposome and a gel precursor; wherein the liposome encapsulates a payload that is capable of inducing gelation of the gel precursor;
b) applying ultrasound to the mixture; to trigger release of the payload from the liposome and induce gelation of the gel precursor.

The payload may act indirectly on the precursor to induce gelation (e.g. by activation of an enzyme, which then acts on the precursor to induce gelation).

The mixture may further comprise a cofactor-dependent enzyme in its inactive form; and the payload may be a cofactor that is capable of activating the enzyme; wherein applying ultrasound to the mixture triggers release of the cofactor from the liposome, which activates the enzyme.

The process for gelation (e.g. hydrogelation) may comprise the steps of:
a) providing a mixture comprising a liposome, a cofactor-dependent enzyme in its inactive form, and a gel precursor; wherein the liposome encapsulates a cofactor that is capable of activating the enzyme;
b) applying ultrasound to the mixture; to trigger release of the cofactor from the liposome and activate the enzyme; to induce gelation of the gel precursor.

The gel precursor may be selected from fibrinogen, collagen, alginate, poly(ethylene glycol), poly(vinyl alcohol), poly(acrylic acid), or a methacrylate-, tetrazine-, or norbornene-modified biopolymer or a mixture thereof. Preferably, the gel precursor may be fibrinogen.

The cofactor may be an ionic cofactor, such as a metal ion. The metal ion may be a divalent or trivalent cation. The metal ion may be a calcium, zinc, iron, magnesium, aluminium, barium or strontium ion, or a combination thereof; preferably a calcium ion.

The enzyme may be a transglutaminase, oxidoreductase, peroxidase, transferase, hydrolase, alcohol dehydrogenase, lyase, isomerase or ligase, or a combination thereof. Preferably, the enzyme may be transglutaminase.

The process may be a process for hydrogelation; such that the gelation is hydrogelation and the gel precursor is a hydrogel precursor.

In some embodiments, the invention provides a process for hydrogelation, wherein the process comprises the steps of:
a) providing a mixture comprising a liposome, transglutaminase in its inactive form, and fibrinogen; wherein the liposome encapsulates a calcium ion;
b) applying ultrasound to the mixture; to trigger release of the calcium ion from the liposome and activate the transglutaminase, to induce hydrogelation of the fibrinogen; optionally, wherein the liposome comprises DPPC and DSPE-PEG₂₀₀₀ biotin.

In a process described herein:
a) the cofactor may be a zinc ion and the enzyme may be an alcohol dehydrogenase, lyase, or hydrolase; or
the cofactor may be a calcium ion and the enzyme may be phospholipase A, acyltransferase, or transglutaminase; or
c) the cofactor may be an iron ion and the enzyme may be an alkaline phosphatase; or
d) the cofactor may be a calcium ion, the enzyme may be transglutaminase and the gel precursor may be fibrinogen; or
e) the cofactor may be a calcium ion, the enzyme may be transglutaminase and the gel precursor may comprise poly(ethylene glycol) (PEG) and hyaluronic acid (HA); or
f) the cofactor may be a calcium ion, the enzyme may be peroxidase, and the gel precursor may comprise tyramine and hyaluronic acid; or
g) the cofactor may be a calcium ion, the enzyme may be phospholipase A and the gel precursor may be a phospholipid; or
h) the cofactor may be a calcium ion, the enzyme may be an acyltransferase and the gel precursor may be a molecule containing an acyl moiety; or
i) the cofactor may be a zinc ion, the enzyme may be an alcohol dehydrogenase and the gel precursor may be an alcohol; or
j) the cofactor may be an iron ion, the enzyme may be an alkaline phosphatase and the gel precursor may be a molecule containing a phosphate moiety.

The mixture may further comprise a crosslinker precursor, which is the substrate of the enzyme. Accordingly, applying ultrasound to the mixture may trigger release of the cofactor, which may activate the enzyme to convert the crosslinker precursor to the crosslinker. The crosslinker may then act on the gel precursor to cause gelation.

The payload may act directly on the precursor to induce gelation. For example, the gel precursor may be a polymer that undergoes gelation in the presence of an ion and the payload may be an ion.

The process for gelation (e.g. hydrogelation) may comprise the steps of:
a) providing a mixture comprising a liposome, and a gel precursor; wherein the liposome encapsulates a payload that is capable of directly inducing gelation of the gel precursor;
b) applying ultrasound to the mixture; to trigger payload release from the liposome and induce gelation of the precursor.

The gel precursor may be alginate, gellan gum, chitosan, pectin, sodium polygalacturonate or carboxylated cellulose nanofibrils, or a mixture thereof, and/or the payload may be an ion (for example, a metal ion or OH⁻). The gel precursor may, preferably, be alginate and the payload may be selected from Ca²⁺, Mg²⁺, Sr²⁺, Ba²⁺, Al³⁺ and Fe³⁺, or a mixture thereof.

In some embodiments, the invention provides a process for hydrogelation, wherein the process comprises the steps of:
a) providing a mixture comprising a liposome and alginate; wherein the liposome encapsulates a calcium ion;
b) applying ultrasound to the mixture; to trigger release of the calcium ion from the liposome and induce gelation of the alginate; optionally, wherein the liposome comprises DPPC and DSPE-PEG2000 biotin.

The mixture may further comprise a liquid vehicle (e.g. water, such as saline solution).

The mixture may further comprise an absorption-increasing material (i.e. a material that increases ultrasonic absorption by the mixture). The absorption-increasing material may be glass microspheres. The glass microspheres may have a diameter of from about 1 to about 100 μm or from about 5 to about 50 μm. The glass microspheres may be solid glass. The glass microspheres may comprise soda lime glass. The absorption-increasing material may be graphite powder. The absorption-increasing material may be aluminium oxide powder.

The liposome may be conjugated to a microbubble.

The liposome may comprise one or more lipid bilayers. The lipid bilayers may comprise one or more phosphatidylcholine. For example, the lipid bilayers may comprise DPPC and DSPC, or a mixture thereof.

The ultrasound may be applied for at least about 1 millisecond. Preferably, ultrasound may be applied for at least about 1 second. The ultrasound may be applied at a frequency of at least about 18 kHz. The ultrasound may be applied at a frequency of at least about 20 kHz. The frequency of the ultrasound may be at least about 1 MHz. The frequency of the ultrasound may be at least about 3 MHz. The frequency of the ultrasound may be at most about 10 MHz. The frequency of the ultrasound may be from about 18 kHz to about 10 MHz. The ultrasound may be focused to a region of at least about 0.5 mm³.

In a second aspect, the invention provides a process for ultrasound-triggered enzyme catalysis, wherein the process comprises the steps of:
a) providing a mixture comprising a liposome, a cofactor-dependent enzyme in its inactive form, and a substrate of the enzyme; wherein the liposome encapsulates a cofactor that is capable of activating the enzyme;
b) applying ultrasound to the mixture; to trigger release of the cofactor from the liposome and activate the enzyme.

The cofactor may be an ionic cofactor, such as a metal ion. The metal ion may be a divalent or trivalent cation. The metal ion may be a calcium, zinc, iron, magnesium, aluminium, barium or strontium ion, or a combination thereof; preferably a calcium ion.

The enzyme may be a transglutaminase, oxidoreductase, peroxidase, transferase, hydrolase, alcohol dehydrogenase, lyase, isomerase or ligase, or a combination thereof. Preferably, the enzyme may be transglutaminase.

The substrate may be a gel precursor and the activated enzyme may induce gelation of the gel precursor. Thus, the process of the second aspect may be a process for gelation as in the first aspect.

The gelation may be hydrogelation and the gel precursor may be a hydrogel precursor.

The gel precursor may be selected from fibrinogen, collagen, alginate, poly(ethylene glycol), poly(vinyl alcohol), poly(acrylic acid), or a methacrylate-, tetrazine-, or norbornene-modified biopolymer or a mixture thereof. Preferably, the gel precursor may be fibrinogen.

In a process described herein, the liposome may comprise DPPC and DSPE-PEG2000 biotin; the cofactor may be a calcium ion; the enzyme may be transglutaminase; and the gel precursor may be fibrinogen.

In a process described herein:
a) the cofactor may be a zinc ion and the enzyme may be an alcohol dehydrogenase, lyase, or hydrolase; or
b) the cofactor may be a calcium ion and the enzyme may be phospholipase A, acyltransferase, or transglutaminase; or
c) the cofactor may be an iron ion and the enzyme may be an alkaline phosphatase; or
d) the cofactor may be a calcium ion, the enzyme may be transglutaminase and the gel precursor may be fibrinogen; or
e) the cofactor may be a calcium ion, the enzyme may be transglutaminase and the gel precursor may comprise poly(ethylene glycol) (PEG) and hyaluronic acid (HA); or
f) the cofactor may be a calcium ion, the enzyme may be peroxidase, and the gel precursor may comprise tyramine and hyaluronic acid; or
g) the cofactor may be a calcium ion, the enzyme may be phospholipase A and the gel precursor may be a phospholipid; or
h) the cofactor may be a calcium ion, the enzyme may be an acyltransferase and the gel precursor may be a molecule containing an acyl moiety; or
i) the cofactor may be a zinc ion, the enzyme may be an alcohol dehydrogenase and the gel precursor may be an alcohol; or
j) the cofactor may be an iron ion, the enzyme may be an alkaline phosphatase and the gel precursor may be a molecule containing a phosphate moiety.

In any of the processes for gelation described herein, the gelation may be hydrogelation and the gel precursor may, therefore, be a hydrogel precursor.

The mixture may further comprise a liquid vehicle (e.g. water, such as saline solution).

The mixture may further comprise an absorption-increasing material. The absorption-increasing material may be glass microspheres. The glass microspheres may have a diameter of from about 1 to about 100 μm or from about 5 to about 50 μm. The glass microspheres may be solid glass. The glass microspheres may comprise soda lime glass. The absorption-increasing material may be graphite powder. The absorption-increasing material may be aluminium oxide powder.

The liposome may be conjugated to a microbubble.

The liposome may comprise one or more lipid bilayers. The lipid bilayers may comprise one or more phosphatidylcholine. For example, the lipid bilayers may comprise DPPC and DSPC, or a mixture thereof.

The ultrasound may be applied for at least about 1 millisecond. Preferably, ultrasound may be applied for at least about 1 second. The ultrasound may be applied at a frequency of at least about 18 kHz. The ultrasound may be applied at a frequency of at least about 20 kHz. The frequency of the ultrasound may be at least about 1 MHz. The frequency of the ultrasound may be at least about 3 MHz. The frequency of the ultrasound may be at most about 10 MHz. The frequency of the ultrasound may be from about 18 kHz to about 10 MHz. The ultrasound may be focused to a region of at least about 0.5 mm³.

In a third aspect, the invention provides a process for the release of a payload from a liposome, wherein the process comprises the step of applying ultrasound to a liposome encapsulating a payload; and the payload is a metal ion.

The metal ion may be a divalent or trivalent cation. The metal ion may be a calcium, zinc, iron, magnesium, aluminium, barium or strontium ion, or a combination thereof; preferably a calcium ion.

The liposome may be present in a mixture that comprises a liquid vehicle (e.g. water, such as saline solution).

The mixture may further comprise an absorption-increasing material. The absorption-increasing material may be glass microspheres. The glass microspheres may have a diameter of from about 1 to about 100 μm or from about 5 to about 50 μm. The glass microspheres may be solid glass. The glass microspheres may comprise soda lime glass. The absorption-increasing material may be graphite powder. The absorption-increasing material may be aluminium oxide powder.

The liposome may be conjugated to a microbubble.

The liposome may comprise one or more lipid bilayers. The lipid bilayers may comprise one or more phosphatidylcholine. For example, the lipid bilayers may comprise DPPC and DSPC, or a mixture thereof.

The ultrasound may be applied for at least about 1 millisecond. Preferably, ultrasound may be applied for at least about 1 second. The ultrasound may be applied at a frequency of at least about 18 kHz. The ultrasound may be applied at a frequency of at least about 20 kHz. The frequency of the ultrasound may be at least about 1 MHz. The frequency of the ultrasound may be at least about 3 MHz. The frequency of the ultrasound may be at most about 10 MHz. The frequency of the ultrasound may be from about 18 kHz to about 10 MHz. The ultrasound may be focused to a region of at least about 0.5 mm³.

In a fourth aspect, the invention provides the use of ultrasound for releasing a payload from a liposome, by applying ultrasound to a liposome encapsulating a payload; wherein the payload is a metal ion.

The metal ion may be a divalent or trivalent cation. The metal ion may be selected from a calcium, zinc, iron, magnesium, aluminium, barium or strontium ion, or a combination thereof. For example, the metal ion may be a calcium ion.

The liposome may be present in a mixture that comprises a liquid vehicle (e.g. water, such as saline solution).

The mixture may further comprise an absorption-increasing material. The absorption-increasing material may be glass microspheres. The glass microspheres may have a diameter of from about 1 to about 100 μm or from about 5 to about 50 μm. The glass microspheres may be solid glass. The glass microspheres may comprise soda lime glass. The absorption-increasing material may be graphite powder. The absorption-increasing material may be aluminium oxide powder.

The liposome may be conjugated to a microbubble.

The liposome may comprise one or more lipid bilayers. The lipid bilayers may comprise one or more phosphatidylcholine. For example, the lipid bilayers may comprise DPPC and DSPC, or a mixture thereof.

The ultrasound may be applied for at least about 1 millisecond. Preferably, ultrasound may be applied for at least about 1 second. The ultrasound may be applied at a frequency of at least about 18 kHz. The ultrasound may be applied at a frequency of at least about 20 kHz. The frequency of the ultrasound may be at least about 1 MHz. The frequency of the ultrasound may be at least about 3 MHz. The frequency of the ultrasound may be at most about 10 MHz. The frequency of the ultrasound may be from about 18 kHz to about 10 MHz. The ultrasound may be focused to a region of at least about 0.5 mm³.

In a fifth aspect, the invention provides a composition comprising a gel precursor and a liposome conjugated to a microbubble, wherein the liposome encapsulates a payload that is capable of inducing gelation of the gel precursor and the liposome comprises a PEGylated lipid.

The composition may further comprise a cofactor-dependent enzyme in its inactive form and the payload may be a cofactor that is capable of activating the enzyme. The gel precursor may be selected from fibrinogen, collagen, and alginate, poly(ethylene glycol), poly(vinyl alcohol), poly(acrylic acid), or a methacrylate-, tetrazine-, or norbornene-modified biopolymer or a mixture thereof. Preferably, the gel precursor may be fibrinogen.

The gel precursor may be a polymer that undergoes gelation in the presence of an ion and the payload may be an ion. The gel precursor may be alginate, gellan gum, chitosan, pectin, sodium polygalacturonate or carboxylated cellulose nanofibrils, or a mixture thereof, and/or the payload may be an ion (for example a metal ion or OH⁻). The gel precursor may, preferably, be alginate and the payload may be selected from Ca²⁺, Mg²⁺, Sr²⁺, Ba²⁺, Al³⁺ and Fe³⁺, or a mixture thereof.

In a composition described herein:
a) the payload may be a zinc ion and the enzyme may be an alcohol dehydrogenase, lyase, or hydrolase; or
b) the payload may be a calcium ion and the enzyme may be phospholipase A, acyltransferase, or transglutaminase; or
c) the payload may be an iron ion and the enzyme may be an alkaline phosphatase; or
d) the payload may be a calcium ion, the enzyme may be transglutaminase and the gel precursor may be fibrinogen; or
e) the payload may be a calcium ion, the enzyme may be transglutaminase and the gel precursor may comprise poly(ethylene glycol) (PEG) and hyaluronic acid (HA); or
f) the payload may be a calcium ion, the enzyme may be peroxidase, and the gel precursor may comprise tyramine and hyaluronic acid; or
g) the payload may be a calcium ion, the enzyme may be phospholipase A and the gel precursor may be a phospholipid; or
h) the payload may be a calcium ion, the enzyme may be an acyltransferase and the gel precursor may be a molecule containing an acyl moiety; or
i) the payload may be a zinc ion, the enzyme may be an alcohol dehydrogenase and the gel precursor may be an alcohol; or
j) the payload may be an iron ion, the enzyme may be an alkaline phosphatase and the gel precursor may be a molecule containing a phosphate moiety.

The composition may further comprise a liquid vehicle (e.g. water, such as saline solution).

The composition may further comprise an absorption-increasing material. The absorption-increasing material may be glass microspheres. The glass microspheres may have a diameter of from about 1 to about 100 μm or from about 5 to about 50 μm. The glass microspheres may be solid glass. The glass microspheres may comprise soda lime glass. The absorption-increasing material may be graphite powder. The absorption-increasing material may be aluminium oxide powder.

The liposome may be conjugated to a microbubble.

The liposome may comprise one or more lipid bilayers. The lipid bilayers may comprise one or more phosphatidylcholine. For example, the lipid bilayers may comprise DPPC and DSPC, or a mixture thereof.

In a sixth aspect, the invention provides a composition comprising an enzyme, a substrate of said enzyme, and a liposome conjugated to a microbubble, wherein the liposome is loaded with a cofactor required to activate said enzyme.

The cofactor may be an ionic cofactor, such as a metal ion. The metal ion may be a divalent or trivalent cation. The metal ion may be selected from a calcium, zinc, iron, magnesium, aluminium, barium or strontium ion, or a combination thereof. For example, the metal ion may be a calcium ion.

The enzyme may be a transglutaminase, oxidoreductase, peroxidase, transferase, hydrolase, alcohol dehydrogenase, lyase, isomerase or ligase, or a combination thereof. Preferably, the enzyme may be transglutaminase.

The substrate may be a gel precursor (e.g. a hydrogel precursor). The gel precursor may be selected from fibrinogen, collagen, and alginate, poly(ethylene glycol), poly(vinyl alcohol), poly(acrylic acid), or a methacrylate-, tetrazine-, or norbornene-modified biopolymer or a mixture thereof. Preferably, the gel precursor is fibrinogen.

The composition may further comprise a liquid vehicle (e.g. water, such as saline solution).

The composition may further comprise an absorption-increasing material. The absorption-increasing material may be glass microspheres. The glass microspheres may have a diameter of from about 1 to about 100 μm or from about 5 to about 50 μm. The glass microspheres may be solid glass. The glass microspheres may comprise soda lime glass. The absorption-increasing material may be graphite powder. The absorption-increasing material may be aluminium oxide powder.

The liposome may be conjugated to a microbubble.

The liposome may comprise one or more lipid bilayers. The lipid bilayers may comprise one or more phosphatidylcholine. For example, the lipid bilayers may comprise DPPC and DSPC, or a mixture thereof.

In a seventh aspect, the invention provides a process, use or composition substantially as herein described.

Embodiments described herein in relation to the first aspect of the invention apply mutatis mutandis to the second to seventh aspects of the invention.

### DESCRIPTION OF THE FIGURES

FIG. 1 shows a schematic of ultrasound-triggered (a) enzyme catalysis and (b) hydrogelation.

FIG. 2 shows SANS analysis of DPPC/DSPE-PEG₂₀₀₀ biotin liposomes loaded using 0.4 M calcium chloride. (a) Unextruded and (b) extruded liposomes were analyzed using SANS (markers) and fitted to a lamellar model (line).

FIG. 3 shows representative cryo-TEM images of DPPC/DSPE-PEG₂₀₀₀ biotin liposomes loaded using 0.4 M calcium chloride. (a) Unextruded and (b) extruded liposomes were imaged using cryo-TEM. Scale bars: 200 nm.

FIG. 4 shows sizing analysis of DPPC/DSPE-PEG₂₀₀₀ biotin liposomes loaded using 0.4 M calcium chloride. (a) DLS measurements (b) NTA measurements.

FIG. 5 shows the effect of CaCl₂ concentration during lipid hydration. (a) NTA particle counting was used to measure the yield of liposomes hydrated using different CaCl₂ solutions. (b) An o-CPC assay was used to quantify the calcium loading into liposomes hydrated using different CaCl₂ solutions, with this value normalized by the number of liposomes.

FIG. 6 shows calcium leakage from DPPC/DSPE-PEG₂₀₀₀ biotin liposomes loaded using 0.4 M calcium chloride. Calcium-loaded liposomes were incubated at 25° C. for 5 d, with the released calcium measured at intervals using an o-CPC assay.

FIG. 7 shows ultrasound-triggered enzyme catalysis and hydrogelation using calcium-loaded liposomes. (a) Calcium-loaded liposomes were exposed to ultrasound for 0-50 s, with the released calcium quantified using an o-CPC assay. (b) The enzymatically-catalyzed conversion of dansylcadaverine was measured after calcium-loaded liposomes were exposed to ultrasound for 0-50 s (c) The rate of dansylcadaverine conversion was measured as a function of ultrasound exposure. The transglutaminase-catalyzed hydrogelation of fibrinogen was measured using time-sweep rheology after the application of 3 (d), 10 (e) or 50 (f) s ultrasound.

FIG. 8 shows a rheology control experiment for liposomes with no ultrasound exposure. (a) Frequency and (b) strain sweeps were performed on solutions of calcium-loaded liposomes, transglutaminase and fibrinogen that had not been exposed to ultrasound (measured after 6 h).

FIG. 9 shows ultrasound-triggered transglutaminase catalysis. 21 h endpoint measurements of the bound dansylcadaverine after transglutaminase, dansylcadaverine and calcium-loaded liposomes were exposed to ultrasound for 0, 1, 3 and 5 s.

FIG. 10 shows size analysis of DSPC/DSPE-PEG₂₀₀₀ /DSPE-PEG₂₀₀₀ biotin microbubbles. (a) Bright field microscopy. Scale bar: 20 μm. (B) Average-shifted histogram showing the diameter distribution of the microbubbles, as determined by bright field image analysis of 890 microbubbles.

FIG. 11 shows ultrasound-triggered hydrogelation using calcium-loaded microbubble-liposome conjugates. (a) Schematic of the microbubble-liposome conjugation. (b) Confocal fluorescence microscopy showing Dil-labelled microbubbles (shown in yellow) colocalized with DiO-labelled liposomes (shown in blue). Scale bar: 20 μm. (c) Superresolution Z-projection of DiO-labelled liposomes (shown in blue) conjugated to a single microbubble obtained using structure illumination microscopy. Scale bar: 3 μm. (d) Camera images and bright field microscopy showing intact microbubble-liposome conjugates before and after ultrasound exposure. Scale bar: 20 μm. (e) The percentage of released calcium measured from dose-matched liposomes and liposome-microbubble conjugates after ultrasound exposure (20 kHz, 25% duty cycle, 20% amplitude, 5 s). (f) Frequency and (g) strain sweeps of the fibrinogen hydrogel obtained after 5 s ultrasound exposure and 42 h static gelation. (h) Picture of a fibrinogen hydrogel, 42 h after the calcium-loaded liposome-microbubble conjugates were exposed to ultrasound.

FIG. 12 shows microbubble-liposome conjugation. The total calcium was measured using an o-CPC assay and then normalized by the microbubble-liposome concentration.

FIG. 13 shows a control experiment for microbubble-liposome conjugates with no ultrasound exposure. (a) Frequency and (b) strain sweeps were performed on solutions of calcium-loaded microbubble-liposome conjugates, transglutaminase and fibrinogen that had not been exposed to ultrasound (measured after 42 h). (c) Picture of control fibrinogen solution, with no ultrasound applied to the calcium-loaded microbubble-liposome conjugates. Image captured after 42 h.

FIG. 14 shows the percentage of released calcium from liposomes upon incubation at different temperatures.

FIG. 15 shows temperature monitoring (top) and passive cavitation detection (bottom) when ultrasound (1.1 MHz, 72% duty cycle, 65 mVₚₚ) was applied for 5 min to a mixture of calcium-loaded liposomes and alginate.

FIG. 16 shows a one-pot ultrasound-triggered fibrinogen hydrogelation. Ultrasound was applied for 10 s to a mixture of fibrinogen, calcium-loaded liposomes and transglutaminase.

FIG. 17 shows ultrasound-triggered fibrinogen hydrogelation with varying transglutaminase concentration. Calcium-loaded liposomes were exposed to ultrasound for 50 s and the gelation of fibrinogen was measured using time-sweep rheometry upon the addition of (a) 1.25 μM, (b) 5 μM and (c) 10 μM transglutaminase.

FIG. 18 shows ultrasound-triggered hydrogelation with varying fibrinogen concentration. Calcium-loaded liposomes were exposed to ultrasound for 50 s and the gelation of an (a) 11.2 mg mL⁻¹, (b) 22.4 mg mL⁻¹ and (c) 33.6 mg mL⁻¹ fibrinogen solution was measured using time-sweep rheometry.

FIG. 19 shows ultrasound-triggered fibrinogen hydrogelation with increased crosslinking time using a 33.6 mg mL⁻¹ fibrinogen solution. The gelation was measured using time sweep rheology after the application of ultrasound for 50 s to calcium-loaded liposomes.

FIG. 20 shows (a) frequency (b) and strain sweeps of alginate hydrogels obtained by exposing a mixture of 2 wt/v % alginate, calcium-loaded liposomes and 6 v/v % glass microspheres to ultrasound operated at 1.1 MHz.

FIG. 21 shows (a) frequency (b) and strain sweeps of alginate hydrogels obtained by exposing a mixture of 2 wt/v % alginate, calcium-loaded liposomes and 6 v/v % glass microspheres to ultrasound operated at 3.3 MHz.

### DETAILED DESCRIPTION

The present disclosure relates to ultrasound-triggered liposome payload release and its use in, for example, the formation of hydrogels through ultrasound-triggered gelation. Further, the present disclosure relates to ultrasound-triggered enzyme catalysis, for example the formation of hydrogels through ultrasound-triggered enzymatic gelation.

One potentially valuable trigger for gelation is ultrasound: mechanical pressure waves that oscillate at high frequency (approximately 18 kHz and above, for example approximately 20 kHz and above) and may produce a range of thermal and non-thermal effects. For example, the absorption of ultrasonic energy by the surrounding medium can produce localized hyperthermia and acoustic streaming, while ultrasound pressure oscillations can generate acoustic radiation forces and modulate the nucleation, growth and oscillation of gaseous microbubbles. These effects have been exploited for a variety of biomedical applications: to pattern cell arrays for in vitro tissue engineering, stimulate osteogenesis for accelerated bone fracture healing, temporarily disrupt the blood-brain-barrier for systemic drug delivery, induce localized hyperthermia for ablation therapy, and to visualize anatomical structure and blood perfusion using ultrasonography.

Described herein is ultrasound-triggered gelation, which is achieved via ultrasound-triggered release of a payload encapsulated in a liposome, wherein the payload is capable of inducing gelation of a gel precursor (for example, a hydrogel precursor). The payload may act directly on the gel precursor to induce gelation. Alternatively, the payload may act indirectly on the gel precursor to induce gelation (e.g. by activation of an enzyme). Thus, the process may be a process for ultrasound-modulated enzyme catalysis. For example, ultrasound may be used to release a cofactor (such as calcium ions) encapsulated in liposomes in order to activate a cofactor dependent enzyme (such as transglutaminase). The ultrasound-activated enzyme can then catalyze intermolecular covalent crosslinking between gel precursor molecules to form a gel (for example, crosslinking between the lysine and glutamine sidechain residues of soluble fibrinogen molecules, in order to produce fibrinogen hydrogels).

Such processes may provide a high degree of control over the gel formation, with the cofactor release, catalysis rate and gelation rate dependent upon the ultrasound exposure time.

Overall, these processes may enable on-demand, ultrasound-triggered gelation without the use of radical species or stimuli-responsive polymers. Indeed, the underlying principles are readily applicable to a range of cofactor-dependent enzymes and/or gel systems. This versatility presents a host of opportunities for in vitro and in vivo applications in material science, biomedical engineering, drug delivery and beyond.

Further, the present invention provides a new approach to achieve ultrasound-triggered enzyme catalysis, as demonstrated by ultrasound-triggered enzymatic gelation. The use of ultrasound represents an entirely new class of stimuli for enzyme activity and gelation that sit alongside the traditional triggers of light, pH, temperature and chemical addition. While transglutaminase was used as an exemplar in this work, the same principles could be applied to other enzymes with cofactors, which include many oxidoreductases, transferases, hydrolases, lyases, isomerases and ligases.

The versatility of this technique extends beyond fibrinogen hydrogelation, opening up a wide range of opportunities for ultrasound-triggered molecular biology, synthetic biology and material science.

Accordingly, in a first aspect, the disclosure provides a process for gelation, wherein the process comprises the steps of:
a) providing a mixture comprising a liposome and a gel precursor; wherein the liposome encapsulates a payload that is capable of inducing gelation of the gel precursor;
b) applying ultrasound to the mixture; to trigger release of the payload from the liposome and induce gelation of the gel precursor.

“Gelation” refers to the formation of a gel from a gel precursor. A gel precursor is a polymer, mixture of polymers or mixture of polymers and monomers that are able to undergo cross-linking to form a gel. The gelation referred to herein is preferably hydrogelation.

“Hydrogelation” refers to the formation of a hydrogel. Hydrogels are hydrated, three-dimensional polymeric networks capable, for example, of absorbing and retaining large quantities of water to form a stable structure. Hydrogels may be formed through crosslinking of hydrogel precursor molecules.

“Hydrogel precursor” refers to a polymer or mixture of polymers and/or monomers that is capable of forming a hydrogel.

The gel or hydrogel precursor may be a polymer that undergoes gelation in the presence of an ion (e.g. a metal ion such as Ca²⁺). The gel or hydrogel precursor may be a polymer that undergoes gelation in the presence of an enzyme (e.g. an activated cofactor-dependent enzyme).

The gel or hydrogel precursor may be a hydrophilic homopolymer, copolymer or macromer. The gel or hydrogel precursor may be a naturally-occurring polymer (e.g. a polysaccharide), a fully synthetic polymer (e.g. poly(ethylene glycol), poly(vinyl alcohol), poly(acrylic acid)) or a semi-synthetic polymer (e.g. methacrylate-, tetrazine-, or norbornene-modified biopolymers), or a combination thereof.

The methacrylate-, tetrazine-, or norbornene-modified biopolymer may be a polynucleotide (such as DNA and RNA), polypeptide or polysaccharide that has been modified with a methacrylate, tetrazine or norbornene moiety.

The gel or hydrogel precursor may be a naturally occurring polymer selected from fibrinogen, collagen, alginate, or a combination thereof. In particular, the gel or hydrogel precursor may be fibrinogen.

The gel or hydrogel precursor may be a synthetic polymer such as a poly(ethylene glycol) (PEG)- or hyaluronic acid (HA)-based polymer. A natural or synthetic polymer may be functionalised with peptide(s) (e.g. having glutamine and lysine residues), such that the polymer undergoes gelation in the presence of an enzyme (e.g. transglutaminase). For example, PEG- or HA-based polymers may be functionalised with two different peptide sequences, one containing glutamine residues and one containing lysine residues to obtain transglutaminase-crosslinked PEG-HA hydrogels (see, for example, Biomacromolecules, 2016, 175), 1553-1560, which is incorporated by reference herein in its entirety).

The precursor polymers may be able to self-crosslink (i.e. a cross-link may be able to form between two of the same polymer molecules). For example, fibrinogen is able to self-crosslink. Alternatively, one polymer may be functionalised with one functional group and another polymer may be functionalised with another polymer and, thus, the gel precursor should comprise a mixture of polymers. See, for example, the hydrogels discussed in A. Ranga et al., Biomacromolecules, 2016, 17, 5, 1553-1560, the entire contents of which are herein incorporated by reference. For example, the payload may be itself be a gel precursor that is capable of inducing gelation of the gel precursor present in the mixture. Thus, the mixture may contain a first gel precursor and the payload may be a second gel precursor, wherein gelation only occurs when the first and second precursors are combined. Accordingly, when ultrasound is applied to the mixture and the payload is released from the liposome into the mixture, gelation occurs.

Reference to, for example, fibrinogen, denotes this polymer as being in its non-gelated (e.g. liquid) form (i.e. prior to (hydro)gelation). Once gelation has taken place, this polymer is referred to as fibrinogen (hydro)gel.

Gelation may be monitored using time-resolved rheology with a rheometer. Gelation occurs when the elastic modulus (G′) exceeds the viscous modulus (G″). In some embodiments, the elastic modulus (G′) may exceed the viscous modulus (G″) within the first 30 minutes following ultrasound exposure. The elastic and viscous moduli G′ and G″ may be determined, for example, by performing a time sweep experiment over 5 h at 1% strain and 1 rad s⁻¹ with an AR 2000 rheometer (TA instruments) equipped with an 8 mm steel parallel plate and an oil chamber to prevent solvent evaporation. The sample may be loaded on the rheometer plate and the 8 mm steel parallel plate (upper plate) may be lowered to have a gap of 1 mm. Measurements may be taken at 1% strain and 1 rad s⁻¹ over time. The output values from the rheometer are the elastic and viscous moduli G′ and G″.

The gelation according to the first aspect may be controllable, through user-defined exposure of the liposome to ultrasound. The use of ultrasound may allow spatiotemporal control of gelation. The cofactor release, enzyme kinetics and gelation rate may be tuned by varying the ultrasound exposure time. For gelation induced via activation of an enzyme, gelation rate may also be tuned by varying the concentration of the enzyme. The mechanical properties of the gel may be tuned by varying the concentration of the gel precursor.

The payload may act directly on the gel precursor to induce gelation (e.g. as a catalyst or reagent). For example, alginate undergoes gelation when mixed with small divalent cations, such as Ca²⁺, Mg²⁺, Sr²⁺ and Ba²⁺, or trivalent cations such as Al³⁺ or Fe³⁺. Additionally, gellan gum may gel in presence of ions or with NaCl. Chitosan may form gels in the presence of OH⁻ ions (see J. Nie et al., Nature Scientific Reports, 2016, 6, 36005, the entire contents of which are herein incorporated by reference). Pectin is a polysaccharide that gels with calcium ions. Sodium polygalacturonate is an anionic linear homopolymer which gels with calcium, zinc, barium and magnesium ions (U. Huynh et al., Carbohydrate Polymers, 2018, 190, 121-128, the entire contents of which are herein incorporated by reference). Carboxylated cellulose nanofibrils can form gels with Ca²⁺, Zn²⁺, Cu²⁺, Al³⁺, and Fe³⁺ (H. Dong et al., Biomacromolecules, 2013, 14, 9, 3338-3345, the entire contents of which are herein incorporated by reference). Thus, described herein is a process for gelation (e.g. hydrogelation), wherein the process comprises the steps of:
a) providing a mixture comprising a liposome, and a gel precursor; wherein the liposome encapsulates a payload that is capable of directly inducing gelation of the gel precursor (for example, the gel precursor may be a polymer that undergoes gelation in the presence of an ion and the payload may be an ion);
b) applying ultrasound to the mixture; to trigger release of the payload from the liposome and induce gelation of the precursor.

The gel precursor may be alginate, gellan gum, chitosan, pectin, sodium polygalacturonate or carboxylated cellulose nanofibrils, or a mixture thereof, and the payload may be an ion. The gel precursor may, preferably, be alginate and the payload may be selected from Ca²⁺, Mg²⁺, Sr²⁺, Ba²⁺, Al³⁺ and Fe³⁺, or a mixture thereof. Alternatively, the gel precursor may be gellan gum and the payload may be selected from a metal ion or NaCl. The gel precursor may be chitosan and the payload may be OH⁻. The gel precursor may be pectin and the payload may be Ca²⁺. The gel precursor may be sodium polygalacturonate and the payload may be selected from calcium, zinc, barium and magnesium ions, or a mixture thereof. The gel precursor may be carboxylated cellulose nanofibrils and the payload may be selected from Ca²⁺, Zn²⁺, Cu²⁺, Al³⁺, and Fe³⁺, or a mixture thereof.

The payload may act indirectly on the gel precursor to induce gelation (e.g. by activation of an enzyme).

In the process according to the first aspect, the mixture may further comprise a cofactor-dependent enzyme in its inactive form; and the payload may be a cofactor that is capable of activating the enzyme. Accordingly, applying ultrasound to the mixture may trigger release of the cofactor, which activates the enzyme. The enzyme may then act on the gel precursor to catalyse gelation.

Thus, described herein is a process for gelation (e.g. hydrogelation), wherein the process comprises the steps of:
a) providing a mixture comprising a liposome, a cofactor-dependent enzyme in its inactive form, and a gel precursor; wherein the liposome encapsulates a cofactor that is capable of activating the enzyme;
b) applying ultrasound to the mixture; to trigger release of the cofactor from the liposome and activate the enzyme. This, therefore, triggers action of the enzyme on the gel precursor resulting in gelation of the gel precursor.

It will be appreciated that the gel (e.g. hydrogel) precursor is the substrate of the enzyme. Thus, activation of the enzyme triggers action of the enzyme on the gel precursor substrate to catalyse the formation of the gel.

The substrate of the enzyme refers to any molecule upon which that enzyme acts (e.g. wherein the enzyme catalyses a chemical reaction involving the substrate).

Step b) of applying ultrasound to the mixture to trigger cofactor release from the liposome and activate the enzyme results in gelating (e.g. hydrogelating) the gel (e.g. hydrogel) precursor through action of the activated cofactor-dependent enzyme on the gel precursor, to obtain a gel (e.g. hydrogel).

Alternatively, in the process according to the first aspect, the mixture may further comprise an enzyme and a crosslinker precursor, which is the substrate of the enzyme. Accordingly, applying ultrasound to the mixture may trigger release of the cofactor, which may activate the enzyme to convert the crosslinker precursor to the crosslinker. The crosslinker may then act on the gel precursor to cause gelation.

Alternatively, in the process according to the first aspect, the mixture may further comprise an enzyme; and the payload may be a crosslinker precursor which is a substrate of the enzyme. Accordingly, applying ultrasound to the mixture may trigger release of the crosslinker precursor, which may be converted to the crosslinker by the enzyme. The crosslinker may then act on the gel precursor to cause gelation.

In a modified process for gelation (e.g. hydrogelation), the liposome encapsulates a cofactor-dependent enzyme in its inactive form and the mixture comprises a liposome, a cofactor that is capable of activating the enzyme, and a gel precursor. Accordingly, the modified process comprises the steps of:
a) providing a mixture comprising a liposome, a cofactor, and a gel precursor; wherein the liposome encapsulates a cofactor-dependent enzyme in its inactive form; and the cofactor is a cofactor that is capable of activating the enzyme;
b) applying ultrasound to the mixture; to trigger release of the enzyme from the liposome so that it is activated by the cofactor; to induce gelation of the gel precursor.

In a second aspect, the disclosure provides a process for ultrasound-triggered enzyme catalysis, wherein the process comprises the steps of:
a) providing a mixture comprising a liposome, a cofactor-dependent enzyme in its inactive form, and a substrate of the enzyme; wherein the liposome encapsulates a cofactor that is capable of activating the enzyme;
b) applying ultrasound to the mixture; to trigger release of the cofactor from the liposome and activate the enzyme.

Step b) of applying ultrasound to the mixture to trigger release of the cofactor from the liposome and activate the enzyme enables catalysis of a reaction involving the substrate through action of the activated cofactor-dependent enzyme on the substrate.

The substrate may be a gel precursor and the activated enzyme may induce gelation of the gel precursor. This process may be a process for gelation. Preferably, the gelation is hydrogelation and the gel precursor is a hydrogel precursor.

“Liposome” refers to a vesicle having at least one lipid bilayer surrounding a cavity (e.g. an aqueous cavity). Preferably, the liposome according to the invention is a unilamellar liposome. More preferably, the liposome is a small unilamellar liposome, for example having an average hydrodynamic diameter of less than about 1000 nm, preferably less than about 500 nm, preferably less than about 200 nm (for example, about 50 to about 200 nm, preferably about 100 to about 200 nm).

The liposomes may be further characterized using dynamic light scattering (DLS), in order to obtain the hydrodynamic diameter of the liposome. As used herein, the average hydrodynamic diameter refers to the z-average of a distribution of sizes measured by dynamic light scattering (DLS) using a light scattering detector. Measurements may be made using a Malvern ZetaSizer, with normalised intensity, volume and number distribution reported as a function of the hydrodynamic diameter.

Liposome diameter may also be measured using nanoparticle tracking analysis (NTA).

Small liposomes are generally preferred for use herein as large liposomes may obstruct vessels in circulation or undergo margination effects, whereas small liposomes (for example, less than about 200 nm) may circulate freely in a cell-free layer of the vessel. In general, small liposomes may exhibit longer circulation half-lives compared to micron-sized particles. See, for example, E. Blanco et al., Nature Biotechnology, 2015, 33, 9, 941-951, the entire contents of which are herein incorporated by reference.

The liposome comprises at least one lipid bilayer, each of which may be independently formed from one or more lipids. The lipid may be selected one of more phosphatidylcholine. The lipid may be a PEGylated lipid (e.g. a PEGylated phosphatidylcholine). The presence of a PEGylated lipid may aid in preventing liposome aggregation. “PEGylated lipid” refers to a lipid that has been modified with polyethylene glycol (PEG).

The lipid may be selected from 1,2-dipalmitoyl-sn-glycero-3-phosphocholine (DPPC), 1,2-d istearoyl-sn-g lycero-3-phosphoethanolamine-N4-[biotinyl(polyethylene glycol)-2000] (DSPE-PEG2000 biotin), or a combination thereof. In some embodiments, the liposome comprises at least one biotinylated lipid (e.g. DSPE-PEG₂₀₀₀ biotin). In further embodiments, the lipid may comprise DPPC and DSPE-PEG₂₀₀₀ biotin. In some embodiments the lipid comprises no more than about 10% DSPE-PEG2000 biotin (e.g. no more than about 5% or no more than about 1% DSPE-PEG2000 biotin).

The mixture may further comprise a liquid vehicle (e.g. water, such as saline solution).

“Cofactor” refers to a molecule that is required by an enzyme for its activity. A cofactor binds with an associated enzyme, which is functionally inactive in the absence of the cofactor, to form the active enzyme. An enzyme that requires a cofactor for its activity may be referred to as a “cofactor-dependent enzyme”. The functionally inactive enzyme may be referred to as an “apoenzyme”. The active enzyme may be referred to as a “holoenzyme”. As would be appreciated by a skilled person, in processes described herein, the cofactor is the cofactor capable of activating the specific enzyme (i.e. the cofactor is complementary to the enzyme).

The mixture provided in the first and second aspects may contain more than one cofactor (e.g. at least two cofactors).

It will be appreciated that reference herein to “activation” of an enzyme includes modulation of the activity of the enzyme such that the activity is increased. Reference to an “enzyme in its inactive form” includes an enzyme in a low-activity state, wherein addition of a cofactor complementary to the enzyme increases its activity, such that the enzyme is in a higher-activity, activated state.

The cofactor may be encapsulated within the liposome (i.e. the liposome is loaded with cofactor). The cofactor may be referred to as the liposome payload. The cofactor-loaded liposomes are stable liposomes that may release their payload upon user-defined ultrasound exposure.

The cofactor may be an ionic co-factor. The ionic cofactor may be a metal ion. The metal ion may be a divalent or trivalent cation. The metal ion may be a calcium, zinc or an iron ion. In some embodiments, the ionic cofactor is a calcium ion (i.e. Ca²⁺). Preferably, an ionic cofactor is encapsulated within the cavity of the liposome.

The cofactor may be a coenzyme. For example, the cofactor may be coenzyme A, a quinone or a vitamin. The coenzyme may be encapsulated within the cavity of the liposome. Alternatively, the coenzyme may be encapsulated by forming part of the liposome lipid bilayer.

The cofactor-dependent enzyme in its inactive form may alternatively be referred to as an apoenzyme. The cofactor-dependent enzyme may be a transglutaminase, oxidoreductase, peroxidase, transferase, hydrolase, alcohol dehydrogenase, lyase, isomerase or ligase. In particular, the enzyme may be a transglutaminase. Transglutaminases are a class of enzymes that catalyze isopeptide bond formation between the E-amine of lysine and the sidechain amide of glutamine. Calcium ions play a key role in binding to transglutaminase and causing a conformational change in the enzyme structure, which exposes an active-site cysteine that can then initiate isopeptide bond formation, which can result in hydrogelation. Enzymes that belong to the transglutaminase family may include plasma-derived Factor XIII, which requires thrombin and calcium to be activated, and tissue transglutaminase (tTGase). Herein, tissue transglutaminase is used as an example. For example, if the enzyme is Factor XIII, thrombin is also required for enzyme activation and may be present in the mixture.

In some embodiments, the cofactor is a zinc ion and the enzyme is an alcohol dehydrogenase, lyase, or hydrolase. In some embodiments, the cofactor is a calcium ion and the enzyme is phospholipase A, acyltransferase, or transglutaminase. In some embodiments, the cofactor is an iron ion and the enzyme is an alkaline phosphatase (e.g. a microbial alkaline phosphatase).

In some embodiments, the cofactor is a calcium ion, the enzyme is transglutaminase and the hydrogel precursor is selected from a naturally occurring polymer (e.g. fibrinogen). In some embodiments, the cofactor is a calcium ion, the enzyme is transglutaminase and the hydrogel precursor is poly(ethylene glycol) (PEG) and hyaluronic acid (HA), resulting in the formation of a PEG-HA hydrogel. In some embodiments, the cofactor is a calcium ion, the enzyme is peroxidase, and the hydrogel precursor is tyramine and hyaluronic acid, resulting in the formation of a HA-tyramine hydrogel.

In some embodiments, the cofactor is a calcium ion, the enzyme is phospholipase A and the substrate is a phospholipid. In some embodiments, the cofactor is a calcium ion, the enzyme is an acyltransferase and the substrate is a molecule containing an acyl moiety. In some embodiments, the cofactor is a zinc ion, the enzyme is an alcohol dehydrogenase and the substrate is an alcohol. In some embodiments, the cofactor is an iron ion, the enzyme is an alkaline phosphatase and the substrate is a molecule containing a phosphate moiety.

Also disclosed herein is a process for forming the liposome loaded with (i.e. encapsulating) a cofactor. The loaded liposome may be formed from a mixture of its component lipids (when the liposome comprises more than one lipid) and a solution of the cofactor (e.g. an aqueous solution). Therefore, the processes of the first and second aspects may further comprise the initial step of forming the liposome, before step a).

The process for forming the liposome may result in a polydisperse mixture of loaded multilamellar liposomes. These multilamellar liposomes may be extruded to form predominantly unilamellar liposomes. The unilamellar liposomes may be treated with solvent (e.g. ethanol) to induce liposome fusion and bilayer interdigitation. Raising the temperature, for example above 50° C., may generate large unilamellar liposomes, which may then be extruded to form small monodisperse unilamellar liposomes. Preferably, the liposomes used herein are monodisperse.

The liposomes may be analysed using small-angle neutron scattering (SANS) and a lamellar model fit, to determine the thickness of the liposome bilayer. Measurements could be carried out at 25° C. The liposomes may have a bilayer thickness of about 1 to about 10 nm, preferably about 5 nm.

The method for formation of liposomes loaded with a cofactor may be a method that produces liposomes with high loading of the cofactor. For example, the method may result in ionic cofactor loading of at least about 10⁻²² mol liposome⁻¹ (preferably at least about 10⁻²¹ mol liposome⁻¹, at least about 10⁻²⁰ mol liposome⁻¹, at least about 10⁻¹⁹ mol liposome⁻¹, at least about 10⁻¹⁸ mol liposome⁻¹, at least about 10⁻¹⁷ mol liposome⁻¹, at least about 10⁻¹⁶ mol liposome⁻¹, or at least about 10⁻¹⁵ mol liposome⁻¹). In some embodiments, the method for forming the loaded liposomes may be an interdigitation fusion vesicle method (e.g. as described in the Examples herein). Alternative methods include lipid film hydration method or freeze-thaw cycling.

The lipid film hydration method may comprise the steps of: 1) preparing a dried lipid film; 2) hydrating the dried lipid film with an aqueous solution (e.g. an aqueous solution containing ions); and 3) shaking the hydrated film (e.g. on a vortex shaker or with a magnetic stirring bar).

The freeze-thaw cycling method may comprise the steps of: 1) preparing a dried lipid film; 2) hydrating the dried lipid film with an aqueous solution (e.g. an aqueous solution containing ions); and 3) performing a heat-cycle on the hydrated lipid film (e.g. between about −80° C. and about 55° C.).

In both the lipid film hydration method and the freeze-thaw cycling method, the temperature at which the lipid suspension is shaken or the thaw step is performed is higher than the lipid transition temperature (Tₘ). The transition temperature determines the phase in which the lipid bilayer is. Below the transition temperature the lipid bilayer is in the gel phase, above the transition temperature the lipid bilayer is in the liquid crystalline phase.

The process for forming the liposomes may be carried out in an organic solvent-water mixture. This may lead to the formation of inverted micelles enclosing an aqueous core encapsulating payload and dispersed in organic solvent. These micelles may be used to form organogels (i.e. gels in which the solvent is an organic solvent). (See for example, Journal of Controlled release, 271, 1-20, which is incorporated by reference herein in its entirety). Phospholipids may be used to form inverted micelles. Unlike liposomes, inverted micelles do not have a bilayer structure. Inverted micelles may have the head group of the phospholipid at the centre and the phospholipid tail extending out. This may result in formation of an aqueous cavity within the micelle.

The cofactor may be a calcium ion and the cofactor solution may be aqueous CaCl₂. In some embodiments, the cofactor solution may be aqueous CaCl₂, when the concentration of CaCl₂ is 0.1 to 1 M, preferably 0.3 to 0.5 M.

When the cofactor is a calcium ion, the liposomal loading may be measured using an ortho-cresolphthalein complexone (o-CPC) colorimetric assay and NTA particle counting. This may be used to determine the most appropriate concentration of ionic cofactor solution to use in the formation of the loaded liposome.

The loading of the liposomes may result in a payload (for example, a cofactor) concentration of at least 50 μM in the mixture.

For other cofactors, suitable assays may be selected for the particular cofactor used. Such assays would be known to a skilled person and are based on the formation of a complex between the ion and a dye, which gives a characteristic change in the absorbance/fluorescence spectrum which depends on the ion concentration.

The concentration of ionic cofactor may also be determined by inductively coupled plasma mass spectrometry (ICP-MS). ICP-MS may be used to measure, for example, calcium, magnesium, iron, barium and zinc ions (see, for example, The Easy Guide to: Inductively Coupled Plasma-Mass Spectrometry (IPC-MS), which is incorporated by reference herein in its entirety).

The mixture according to the process of the first and second aspects may further comprise a liquid vehicle (e.g. water, such as saline solution). The mixture may comprise an organic solvent-water vehicle. Applying ultrasound to the mixture to trigger release of the cofactor (e.g. an ionic cofactor such as a metal ion) from the liposome (or an inverted micelle) may induce gelation of the gel precursor and result in formation of an organogel.

The skilled person will appreciate that the mixture may comprise a plurality of liposomes. When the mixture comprises a liquid vehicle, the mixture may comprise the liposomes, the cofactor-dependent enzyme and the hydrogel precursor at preferred concentrations.

The mixture according to the process of the first and second aspects may further comprise an absorption-increasing material (i.e. a material that increases ultrasonic absorption by the mixture). The presence of an absorption-increasing material may increase the efficiency and control of the triggering process. The absorption-increasing material may be, for example, glass microspheres, graphite powder, and/or aluminium oxide powder.

The absorption-increasing material may be glass microspheres. Glass microspheres would be known to a skilled person. Glass microspheres may be substantially spherical and may have a diameter from about 1 to about 1000 μm. Glass microspheres may be, for example, as described in Mylonopoulou et al, Int. J. Hyperthermia, 2013; 29(2): 133-144, the entire contents of which are herein incorporated by reference. Preferably, the glass microspheres have a diameter of from about 1 to about 100 μm or from about 5 to about 50 μm. The glass microspheres may be solid glass. The glass microspheres may comprise soda lime glass. Glass microspheres may be obtained commercially from Cospheric LLC (e.g. Soda Lime Solid Glass Microspheres 2.5 g/cc 5-50 μm).

The absorption-increasing material may be graphite powder, for example as described in Burlew et al, Radiology, 1980; 134: 517-520, the entire contents of which are herein incorporated by reference.

The absorption-increasing material may be aluminium oxide powder, for example as described Ramnarine et al, Ultrasound in Med. & Biol., 2001; 27(2): 245-250), the entire contents of which are herein incorporated by reference.

Step b), according to the first and second aspects, comprises applying ultrasound to the loaded liposome.

Ultrasound may be applied using a probe sonicator. The probe sonicator may have a tip diameter of about 2 mm, for example as described in the examples.

Alternatively, the ultrasound may be applied using a focused-ultrasound method to trigger gelation in a user defined area. For example, an ultrasonic transducer may be used to apply ultrasound to a specific area to trigger gelation (i.e. such that localised gelation occurs). A skilled person would appreciate that the focal diameter of the transducer would determine the area of ultrasound exposure. For example, a transducer may have a focal diameter of from about 0.5 mm to about 3 mm (e.g. about 1.0 mm, about 1.5 mm, or about 2.0 mm). Thus, gelation may be induced only in the region to which the ultrasound is applied.

Ultrasound may be focused to a region (i.e. to a set volume of the mixture) of at least about 0.5 mm³ (e.g. about 1 mm³).

Ultrasound may be applied for a timeframe, frequency and amplitude that leads to release of at least about 1%, at least about 10%, at least about 25%, at least about 50%, at least about 75%, or at least about 90% of the cofactor from the liposome. When the cofactor is a calcium ion, an o-CPC assay may be performed to quantify the released calcium.

Ultrasound may be applied for at least 1 millisecond. Preferably, ultrasound may be applied for at least 1 second (e.g. 3, 10 or 50 seconds). The frequency of the ultrasound may be at least about 18 kHz, preferably at least about 20 kHz. The ultrasound may be at about 20% amplitude, and about 25% duty cycle. The frequency of the ultrasound may be at least about 1 MHz. The frequency of the ultrasound may be at least about 3 MHz. The frequency of the ultrasound may be at most about 10 MHz. The frequency of the ultrasound may be from about 18 kHz to about 10 MHz. The ultrasound may be about 75% duty cycle. Ultrasound may be applied repeatedly, separated by pre-determined intervals (e.g. two 25 second applications, with a 40 second interval).

The ultrasound may have a pressure amplitude of, for example, at least about 0.5 MPa when the frequency is from about 1 to about 3 MHz.

Activation of the cofactor-dependent enzyme may be monitored using an assay suitable for that enzyme (e.g. transglutaminase activity may be assessed using a dansylcadaverine-based assay).

In some embodiments, liposomes are loaded with calcium ions and the calcium ions are released following exposure of the liposomes to ultrasound. This may be used to trigger the transglutaminase-catalyzed hydrogelation of fibrinogen. Transglutaminase catalyzes intramolecular and intermolecular fibrinogen crosslinking, with the latter used to form fibrinogen hydrogels.

The capabilities of the technology described herein were further extended by conjugating the loaded liposomes to the surface of microbubbles that are commonly used for in vivo drug delivery. These microbubble-liposome conjugates displayed an even greater response to the applied acoustic field and could also be used for ultrasound-triggered elation.

Thus, in some embodiments, the liposome is conjugated to a microbubble. “Microbubble” refers to a gas-filled bubble, preferably having a diameter of no more than about 10 μm.

Conjugation of liposomes to microbubbles is understood to enhance the ultrasound-triggered release of liposomal payload and may increase the efficiency of liposomal payload release.

The microbubble may be a biotinylated microbubble. The microbubble may comprise a fluorocarbon (e.g. perfluorohexane) or air or a mixture thereof, preferably a mixture of perfluorohexane and air.

The microbubble may be prepared by hydrating a lipid film. The lipid film may be formed from a phosphatidylcholine, such as dipalmitoylphosphatidylcholine (DPPC) or 1,2-distearoyl-sn-glycero-3-phosphocholine (DSPC), or a mixture thereof. In some embodiments, the lipid film comprises DSPC, DSPE-PEG or DSPE-PEG₂₀₀₀ biotin, or a mixture thereof. A PEGylated lipid may be present in the lipid film. Alternatively, a cationic lipid may be used to prevent bubble coalescence and/or enhance stability instead of the PEG. Where a PEGylated lipid is present, the lipid film may include at least about 1% PEGylated lipid.

In further embodiments, the lipid film may comprise DSPC, DSPE-PEG and DSPE-PEG₂₀₀₀ biotin, optionally in a molar ratio of about 86:9:5.

The microbubbles may be visualized using bright field microscopy and image analysis to visually determine the arithmetic mean diameter. In some embodiments, the mean microbubble diameter may be about 1 to about 10 μm.

Liposomes may be conjugated to the surface of the microbubbles. In some embodiments, the liposome and microbubble both comprise a lipid with a biotin moiety, said biotin being used to conjugate the liposome and microbubble. The biotin moieties present on the liposome and microbubble may be bound using avidin (for example, neutravidin). Alternatively, conjugation may be carried out using thiol-functionalised microbubbles and thiol-functionalised liposomes as described in Y. Yoon et al., Theranostics, 2014, 4(11), 1133-1144, the entire contents of which are herein incorporated by reference. Maleimide-functionalised liposomes and thiol-functionalised microbubbles may also be used as described in J. M. Escoffre et al., IEEE Transactions on Ultrasonics, Ferroelectrics, and Frequency Control, 2013, 60, 1, the entire contents of which are herein incorporated by reference.

Confocal fluorescence microscopy may be used to confirm conjugation of the liposome and microbubble, by using fluorescently-labelled liposomes and fluorescently-labelled microbubbles. Observation of co-localization of the fluorescently-labelled liposomes on the surface of fluorescently-labelled microbubbles indicates a successful conjugation.

Additionally, structured illumination microscopy (a super-resolution imaging technique) may be used to determine the distribution of liposomes across the microbubble surface. In some embodiments, the liposomes are uniformly distributed across the microbubble surface.

When the cofactor is a calcium ion, an o-CPC calcium assay may be used to measure the loading of calcium ions. The loading of calcium ions may be at least 10⁻¹⁶ mol per conjugate.

Following exposure to ultrasound, the microbubble-liposome conjugates may be evaluated using bright field microscopy and, where the cofactor is a calcium ion, an o-CPC calcium assay. The absence of any microbubble-liposome conjugates after ultrasound exposure indicates widespread destruction of the microbubble population.

In a third aspect, the invention provides a process for the release of a payload from a liposome, wherein the process comprises the step of applying ultrasound to a liposome encapsulating a payload; and the payload is a metal ion.

The metal ion may be a divalent or trivalent cation. The metal ion may be selected from a calcium, zinc, iron, magnesium, aluminium, barium or strontium ion, or a combination thereof. The metal ion may be for use in a downstream application that utilises said metal ion.

In some embodiments, the metal ion is a calcium ion. The calcium ion may be used in a process for gelation or a process for enzyme catalysis, as described herein. The calcium ion may be used to regulate transfection (see, for example, Biochimica et Biophysica Acta (BBA)—Biomembranes, 1463(2), 2000, 279-290, which is incorporated by reference herein in its entirety).

Throughout the description and claims of this specification, the words “comprise” and “contain” and variations of the words, for example “comprising” and “comprises”, mean “including but not limited to”, and are not intended to (and do not) exclude other components. In any of the embodiment described herein, reference to “comprising” also encompasses “consisting essentially of”.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

It will be appreciated that many of the features described above, particularly of the preferred embodiments, are inventive in their own right and not just as part of an embodiment of the present invention. Independent protection may be sought for these features in addition to or alternative to any invention presently claimed.

Reference is now made to the following examples, which illustrate the invention in a non-limiting fashion.

### EXAMPLES

### Materials for Examples 1 to 6

1,2-dipalmitoyl-sn-glycero-3-phosphocholine (DPPC), 1,2-distearoyl-sn-glycero-3-phosphoethanolamine-N-[biotinyl(polyethylene glycol)-2000] (DSPE-PEG₂₀₀₀ biotin), 1,2-distearoyl-sn-glycero-3-phosphocholine (DSPC) and 1,2-distearoyl-sn-g lycero-3-phosphoethanolamine-N-[methoxy(polyethylene glycol)-2000] (ammonium salt) (DSPE-PEG₂₀₀₀) were purchased from Avanti Polar Lipids. All other reagents were purchased from Sigma Aldrich. Ultrapure water (18.2 MΩ cm) was taken from TR Duo10 UF Polisher triple (Triple Red, Avidity Science). Where sonication was carried out using a probe sonicator, the sonicator was a VibraCell VC 750 with 2 mm diameter microtip, Sonics & Materials Inc).

### Example 1: Ultrasound-Triggered Gelation of Fibrinogen Hydrogels

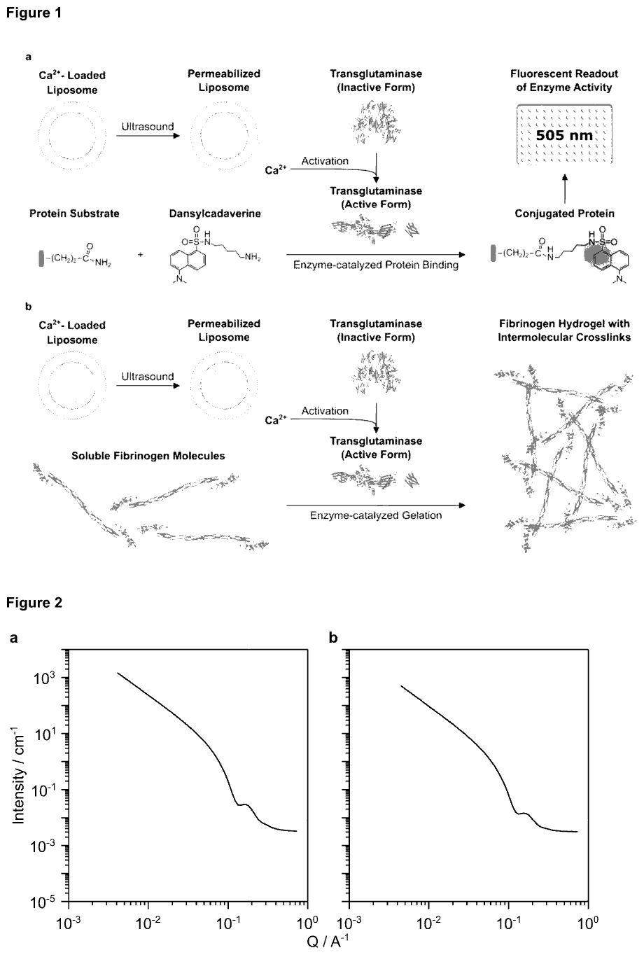

FIG. 1 shows a schematic of ultrasound-triggered enzyme catalysis and hydrogelation. In FIG. 1a: Ultrasound is applied to calcium-loaded liposomes in order to liberate Ca²⁺ ions and activate transglutaminase. The active transglutaminase is then able to catalyze the reaction between a protein substrate and dansylcadaverine. This conjugation process results in a shift of the maximum fluorescence emission wavelength and an increase in fluorescence at 505 nm. In FIG. 1b: A similar process is used to catalyze the crosslinking of soluble fibrinogen molecules. Intermolecular crosslinking results in the formation of fibrinogen hydrogels.

### Liposome Formulation

Calcium-loaded liposomes were formulated using an established interdigitation-fusion vesicle method (Biochim. Biophys. Acta—Biomembr. 1994, 1195, 237). Briefly, a solution of 99 mol % of DPPC and 1 mol % of DSPE-PEG₂₀₀₀ biotin was prepared in chloroform, dried with a stream of nitrogen gas in a glass vial and then kept under vacuum for at least 3 h. The lipid film was hydrated to a lipid concentration of 20 mg mL⁻¹ with an aqueous CaCl₂ solution for 1 h at 55° C. under constant stirring. The liposome solution was extruded 25 times through a 100 nm polycarbonate membrane and 31 times through a 50 nm polycarbonate membrane (Whatman® Nucleopore Track-Etched™ membranes) at 55° C. To induce interdigitation, ethanol was added to a final concentration of 4 M while stirring. The interdigitated gels were stored overnight at 4° C. Five centrifuge washes at 8000 g for 8 min were performed to remove the ethanol, after which the lipid gels were incubated at 55° C. for 2.5 h to form large unilamellar liposomes. These liposomes were then extruded 31 times through a 400 nm polycarbonate membrane (Whatman® Nucleopore Track-Etched™ membranes) at 55° C. to yield a monodisperse population of unilamellar vesicles. The calcium-loaded liposomes were dialyzed against iso-osmotic buffer (0.6 M NaCl) to remove free calcium, and then stored at 4° C. prior to use.

### Small-Angle Neutron Scattering (SANS)

SANS measurements were performed at the SANS2D beamline of the ISIS pulsed neutron source at the Rutherford Appleton Laboratory (Didcot, UK). Samples were loaded in 1 mm path length quartz cuvette cells and measured at 25° C. The source to sample and sample to detector distance was set as L₁=L₂=4 m to give a scattering vector (Q) range of 0.004 to 0.722 Å⁻¹. The scattering angle (θ) was measured for neutrons of wavelengths (λ=1.75-16.5 Å) used simultaneously by time of flight. The scattering vector Q has a modulus of:

$$Q = \frac{4 π}{λ} sin ( \frac{θ}{2} ) . \qquad ( 1 )$$

Data were reduced using MantidPlot (Nucl. Instruments Methods Phys. Res. Sect. A Accel. Spectrometers, Detect. Assoc. Equip. 2014, 764, 156) and the SANS curves were fitted with SasView v4.1.0 (http://www.sasview.org/, Accessed October 2018) using a Lamellar Model. This model describes a lyotropic lamellar phase with uniform scattering length density and random distribution. The 1D scattered intensity I(Q) is:

$$I ( Q ) = φ * \frac{2 π * P ( Q )}{{Q}^{2} * δ} + bkg , \qquad ( 2 )$$

where φ is a scale factor, Q is the modulus of the scattering vector, δ is the total layer thickness and P(Q) is the form factor, defined as:

$$P ( Q ) = \frac{2 Δ {ρ}^{2}}{{Q}^{2}} ( 1 - cos ( Q δ ) ) . \qquad ( 3 )$$

In this case, Δρ is the scattering length density difference. A Gaussian polydispersity function of 15% was used for the bilayer thickness to account for the presence of the PEGylated lipid.

### Cryogenic Transmission Electron Microscopy (Cryo-TEM)

Liposome samples for cryo-TEM were prepared using an automatic plunge freezer (Leica EM GP). Briefly, 4 μL of sample was deposited on QuantiFoil R2/1 copper grids (Electron Microscopy Supplies) in an environmentally-controlled chamber at 90% relative humidity and 20° C. Prior to deposition, the grids were plasma treated (O₂/H₂1:1 for 15 s) using a Gatan SOLARIS plasma cleaner.

After blotting the excess suspension on filter paper, the sample was vitrified in liquid ethane. Samples were stored in liquid nitrogen and imaged at −170° C. using a Gatan 914 cryo-holder in a JEOL 2100Plus transmission electron microscope at 200 kV. Minimum Dose System software was used for imaging, with micrographs acquired using a Gatan Orius SC 1000 camera with a 5 s exposure time, a magnification of 30000 or 15000 and no image binning.

### Liposome Sizing and Quantitation

Samples were prepared for dynamic light scattering (DLS) by dilution to 1.2×10¹² particles mL⁻¹ in iso-osmotic buffer. Measurements were made using a Malvern ZetaSizer, with normalised intensity, volume and number distribution reported as a function of the hydrodynamic diameter. Nanoparticle tracking analysis (NTA) measurements were performed using samples diluted to a concentration of 10⁸-10⁹ particles mL⁻¹ in iso-osmotic buffer. Three 60-s videos were acquired using a NanoSight NS300 at a camera level of 13 and analyzed using NTA V3.0 software with a detection threshold of 5.

### Quantifying Calcium Loading into Liposomes

Liposomes were formulated with either 0.2, 0.4 or 0.6 M aqueous CaCl₂ solutions, as described above. In order to quantify the total encapsulated calcium, the liposomes were lyzed with 5 vol % Triton X-100 at 55° C. for 40 min under stirring and an o-cresolphthalein complexone (o-CPC) assay was then performed. 24.4 μL of each sample was mixed with 24.4 μL of 0.1 M HCl and 132.2 μL of a solution containing 10 mg mL⁻¹ of o-CPC in a sodium borate buffer. The sodium borate buffer was obtained by adding an appropriate volume of an aqueous solution of 2 M NaOH to an aqueous solution of 0.25 M boric acid to have a final pH of 10. The absorbance at 570 nm was measured in a black clear-bottom 96-well half-area plate using a SpectraMax M5 microplate reader. Nanoparticle tracking analysis was used to measure the liposome concentration (see above for full details), which was used to normalize the total encapsulated calcium.

### Quantifiying Liposomal Calcium Leakage

Liposomes prepared with 0.4 M CaCl2 solution were incubated in a 0.6 M NaCl solution at 25° C. over 5 d. Aliquots were taken at different time points and an o-CPC assay performed to measure the free calcium. In order to be within the linear range of the o-CPC assay, the liposomes were diluted to a total encapsulated calcium concentration of 2.55 mM prior to the experiment. A standard curve containing CaCl₂ and liposomes encapsulating 0.6M NaCl, at the same particle concentration of the calcium-loaded liposomes, was used to calculate the calcium in the unknown samples.

### Ultrasound-Triggered Calcium Release from Liposomes

Ultrasound was applied with a probe sonicator (VibraCell) using 20 kHz, 20% amplitude and 25% duty cycle. These parameters were used for all ultrasound triggered studies for examples 1, 2, 4, 5, and 6. Ultrasound was applied to 250 μL of calcium-loaded liposomes in a 500 μL LoBind DNA Eppendorf tube for 1, 3, 5, 10 or 20 s. For the 50 s exposure, ultrasound (20 kHz, 20% amplitude, 25% duty cycle) two 25 s applications were used with a 40 s interval. An o-CPC assay was performed to quantify the released calcium. In order to be within the linear range of the o-CPC assay, the liposomes were diluted to a total encapsulated calcium concentration of 2 mM prior to the experiment. A standard curve of free CaCl₂ in 0.6M NaCl was used to calculate the quantity of calcium in the unknown samples.

### Ultrasound-Triggered Catalysis

Calcium-loaded liposomes were diluted in order to have a total encapsulated calcium concentration of 1 mM. 250 μL aliquots were transferred to a 500 μL LoBind DNA Eppendorf tube, and ultrasound was applied as previously described. Transglutaminase activity was assessed with a dansylcadaverine-based assay. An assay solution was made using dansylcadaverine in 50 mM TRIS-HCl buffer and 25 vol % DMSO, N,N-dimethylcasein, DTT and liposomes sonicated for 0, 1, 3, 5, 10 or 20 s with a probe sonicator. For the 50 s exposure, ultrasound was applied using two 25-s applications with a 40 s interval. The final concentrations of dansylcadaverine, N,N-dimethylcasein and DTT were 47.7 μM, 0.298 mg mL⁻¹ and 2.98 mM, respectively. 7.86 μL of 1.91 μM aqueous transglutaminase solution was added to 142.2 μL of assay mixture in a black clear bottom 96-well half-area plate. The final concentration of transglutaminase was 100 nM. Fluorescence intensity was measured using a SpectraMax M5 microplate reader (ex: 360 nm, em: 505 nm, bottom read) over 21 h, with a cover film used to prevent sample evaporation. For endpoint measurements, ultrasound was applied for 0, 1, 3 or 5 s to a mixture of assay solution and transglutaminase at the same ratios as previously described. Samples were then transferred in a black clear bottom 96-well half-area plate (150 μL/well), covered with a PCR cover film and incubated for 21 h, before measuring the fluorescence intensity using a SpectraMax M5 microplate reader (ex: 360 nm, em: 505 nm, bottom read). All the experiments were performed at 25° C.

### Enzyme Kinetics

A standard curve was used to convert the fluorescence intensity into the concentration of reacted dansylcadaverine. An assay solution was made using dansylcadaverine in 50 mM TRIS-HCl buffer and 25 vol % DMSO, N,N-dimethylcasein and DTT. The final concentrations of dansylcadaverine, N,N-dimethylcasein and DTT were 47.7 μM, 0.298 mg mL⁻¹ and 2.98 mM, respectively. A solution of calcium chloride in 0.6 M NaCl was then added to the mixture to a final concentration of 1 mM, together with transglutaminase to a final concentration of 100 nM. Control samples were prepared by adding an equivalent volume of deionized water in place of the transglutaminase or an equivalent volume of 0.6 M NaCl in place of the calcium chloride solution. After 43 h incubation, a standard curve was prepared by mixing ratios of transglutaminase-containing samples and negative control samples. The fluorescence intensity was measured in a black clear bottom 96-well half-area plate using a SpectraMax M5 microplate reader (ex: 360 nm, em: 505 nm). This enabled the concentration of bound dansylcadaverine to be plotted as a function of time for the ultrasound-triggered catalysis. Using this graph, the gradient of the linear portion of the curves (up to 3 h) was measured and plotted as a function of the ultrasound exposure time. This data was fitted with an asymptotic regression model (R²=0.94) using OriginPro 2017 software.

### Ultrasound-Triggered Hydrogelation using Calcium-Loaded Liposomes

250 μL of calcium-loaded liposomes (total encapsulated calcium of 53.6 mM) was transferred to a 500 μL LoBind DNA Eppendorf tube and sonicated for 0, 3, 10 or 50 s with a probe sonicator. 100 μL of each liposome group were then mixed with DTT in deionized water (final DTT concentration of 8.69 mM) and fibrinogen in 0.6 M NaCl (final fibrinogen concentration of 22.42 mg mL⁻¹). Transglutaminase was then added to a final concentration of 5 μM immediately prior to rheological measurements. A time sweep was performed over 5 h at 1% strain and 1 rad s⁻¹ with an AR 2000 rheometer (TA instruments) equipped with an 8 mm steel parallel plate and an oil chamber to prevent solvent evaporation. The unexposed group was characterized using frequency and strain sweeps. The frequency sweep measurements (0.1 to 100 rad s⁻¹) were performed at 1% strain while the strain sweep measurements (0.1 to 100% strain) were performed at 1 rad s⁻¹. All experiments were performed at 25° C.

### Results and Discussion

Our field-responsive system required a stable formulation of calcium-loaded liposomes that could release their payload upon ultrasound exposure. We selected a liposome formulation consisting of two lipids: 1,2-dipalmitoyl-sn-glycero-3-phosphocholine (DPPC) doped with 1 mol % 1,2-distearoyl-sn-glycero-3-phosphoethanolamine-N-[biotinyl(polyethylene glycol)-2000] (DSPE-PEG2000 biotin). DPPC membranes are in a gel phase at temperatures lower than 41° C., which should provide high cargo retention prior to ultrasound-mediated calcium release. Meanwhile, the small fraction of biotinylated lipid served as a reactive handle for liposome functionalization. We selected an interdigitation-fusion vesicle method in order to produce liposomes with high intraluminal calcium loading (Biochim. Biophys. Acta—Biomembr. 1994, 1195, 237). We hydrated the lipid mixture with aqueous CaCl₂ to produce a polydisperse mixture of calcium-loaded multilamellar liposomes. We used ethanol to induce bilayer interdigitation and generate large unilamellar liposomes, which we then extruded to form small unilamellar liposomes. We analyzed the unextruded and extruded liposomes using small-angle neutron scattering (SANS) and a lamellar model fit, which estimated bilayer thicknesses of 49.1±0.1 Å and 50.9±0.1 Å, respectively (FIG. 2). Data are plotted on a log-log scale.

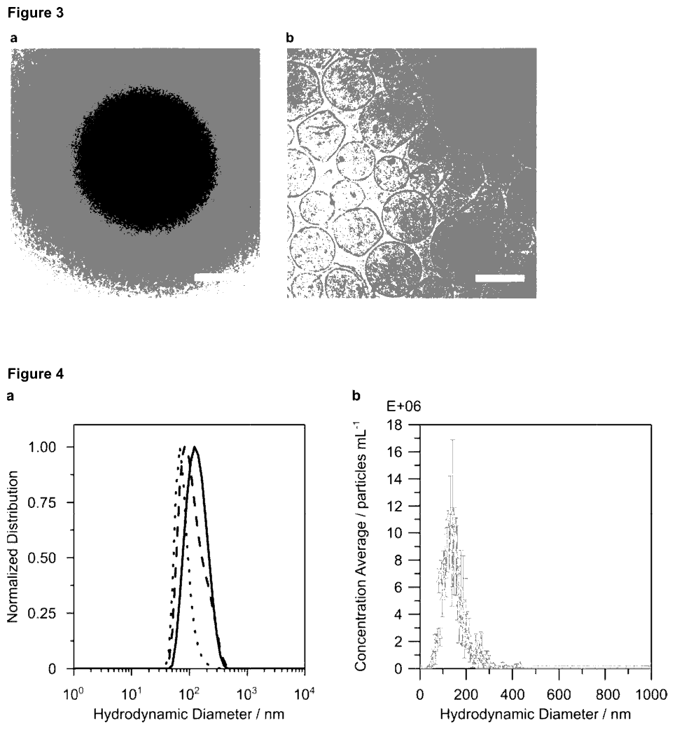

Meanwhile, we used cryogenic transmission electron microscopy (cryo-TEM) to confirm that the liposomes were unilamellar before (FIG. 3a) and after (FIG. 3b) extrusion. The scale bars shown on the cryo-TEM images are 200 nm. We further characterized the extruded liposome population using dynamic light scattering (DLS). DLS measurements showed single peaks for number (dotted line), volume (dashed line) and intensity (black line) distributions, with a z-average hydrodynamic diameter of 122±43 nm and a polydispersity of 0.125 (FIG. 4a). This value correlated well with the liposome diameter of 144±51 nm measured using nanoparticle tracking analysis (NTA) (FIG. 4b).

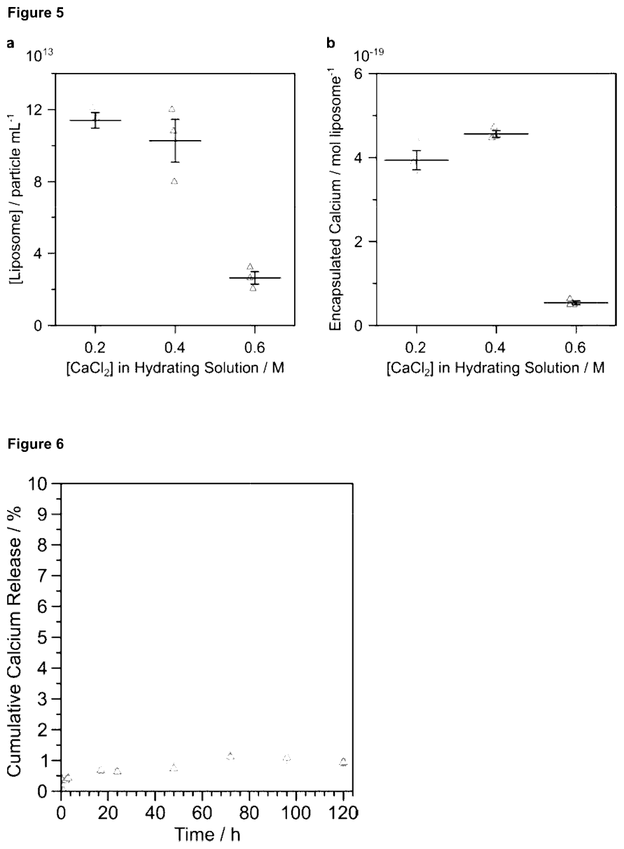

We tested a range of CaCl₂ concentrations during lipid hydration (0.2, 0.4, 0.6 M) and measured the liposomal calcium loading using an ortho-cresolphthalein complexone (o-CPC) colorimetric assay and NTA particle counting (FIG. 5). We observed a 37% increase in the encapsulated calcium per liposome as the concentration was raised from 0.2 M ((3.4±0.3)×10⁻¹⁹ mol liposome⁻¹) to 0.4 M ((4.6±0.1)×10⁻¹⁹ mol liposome⁻¹). However, we also observed a reduced yield of liposomes and a lower calcium loading at the highest tested concentration of 0.6 M CaCl₂ ((0.5±0.06)×10⁻¹⁹ mol liposome⁻¹) (FIG. 5). Data were shown as mean and standard deviation, with data collected from different liposome batches.

Based on these studies, we selected 0.4 M CaCl₂ as the hydrating solution for all subsequent studies. We next investigated the release of calcium from this liposome formulation in the absence and presence of ultrasound, using an o-CPC assay. We observed that our liposomes were stable against uncontrolled calcium leakage, with less than 2% of the encapsulated cargo released after 5 d at 25° C. (FIG. 6). The percentage release was calculated by normalizing the values at each interval to the total calcium level measured from a lysed liposome control. Data shown as mean and standard deviation for three technical replicates using the same batch of liposomes.

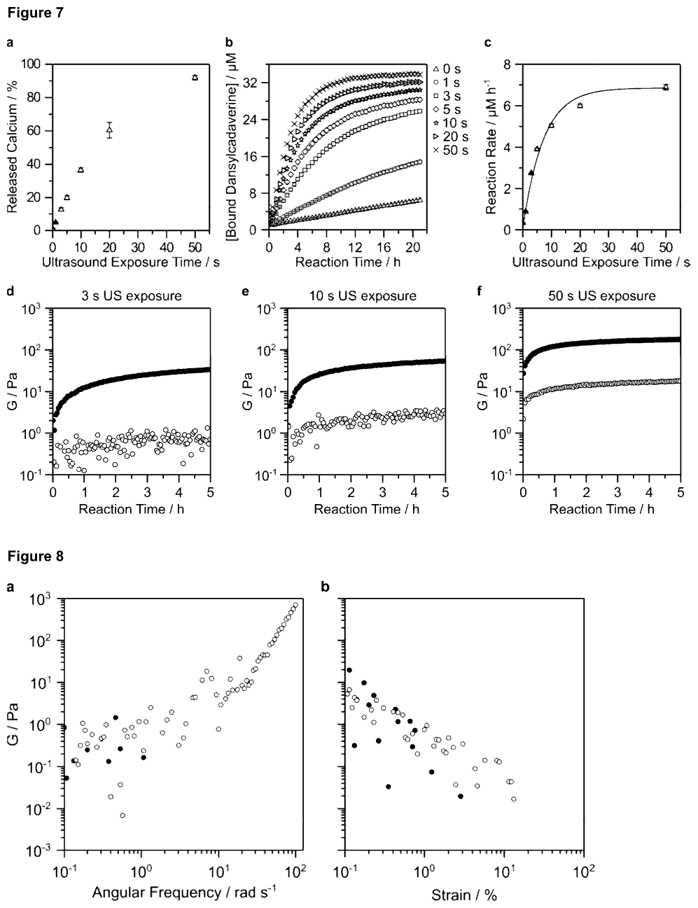

Having established this baseline, we then sought to assess whether we could trigger calcium release from the liposomes using ultrasound. For this study, we applied 20 kHz ultrasound at 20% amplitude and 25% duty cycle, with the exposure time varied between 1 and 50 s. Using these parameters, we were able to liberate up to 92% of the total encapsulated calcium, with a release quantity that was dependent on the ultrasound exposure time (FIG. 7a). Calcium-loaded liposomes were incubated at 25° C. for 5 d, with the released calcium measured at intervals using an o-CPC assay.

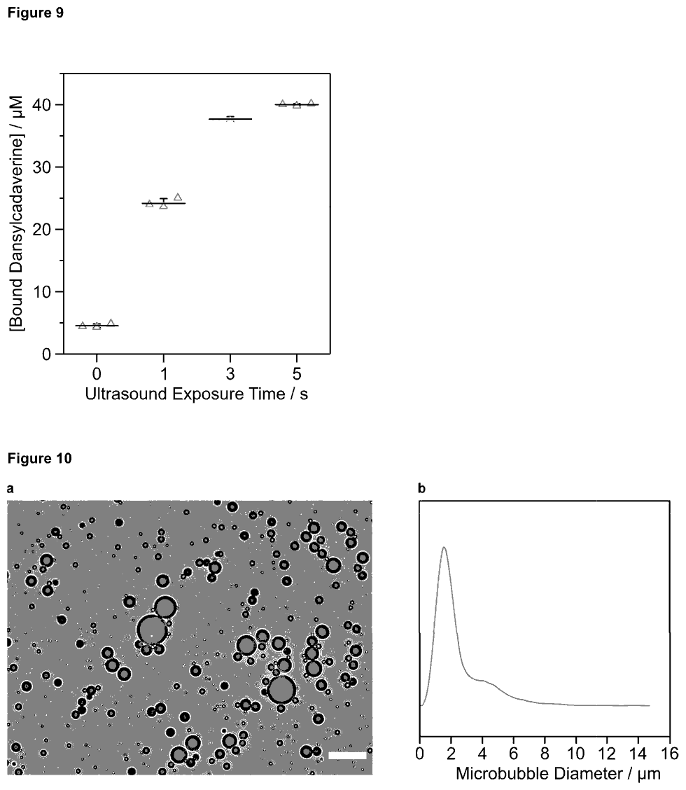

The ability to controllably trigger calcium release using ultrasound opens up a wide range of possible applications. Here, we sought to apply this technology to modulate the catalytic activity of transglutaminase, a calcium-dependent enzyme. The transglutaminases are a class of enzymes that catalyze isopeptide bond formation between the ε-amine of lysine and the sidechain amide of glutamine. Calcium ions play a key role in binding to transglutaminase and causing a conformational change in the enzyme structure, which exposes an active-site cysteine that can then initiate isopeptide bond formation. In order to measure this process, we monitored the fluorescence changes that occurred during the transglutaminase-catalyzed crosslinking between a model protein (N,N-dimethylcasein) and a fluorescent probe (dansylcadaverine). Specifically, we tested whether ultrasound-triggered calcium release could modulate transglutaminase activity over a 21 h period. We observed a dose-dependent enzyme activation when the ultrasound exposure time was varied between 1 and 50 s, and importantly, negligible catalysis without any ultrasound application. The enzymatically-catalyzed conversion of dansylcadaverine was measured after calcium-loaded liposomes were exposed to ultrasound for 0-50 s (FIG. 7b). Data shown are the mean and standard deviation of three technical replicates. The rate of dansylcadaverine conversion was measured as a function of ultrasound exposure (FIG. 7c). Data shown are the mean and standard deviation. We fitted the reaction kinetics to an asymptotic regression model y=a−b*c^{x}, where a=6.85, b=6.78, c=0.87, and R²=0.94. The initial reaction rate increased linearly with increasing ultrasound exposure time, and reached a plateau for 50 s ultrasound exposure, at which 92% of the total encapsulated calcium is released from liposomes. However, it should be noted that in order to measure the early-stage catalytic activity required for kinetic analysis, the transglutaminase was added after exposure and immediately prior to fluorescence monitoring. Nevertheless, end-point fluorescence readings validated that ultrasound could still trigger enzymatic activity in a dose-dependent manner when transglutaminase was present during exposure (FIG. 9). Data are shown as mean and standard deviation of three technical replicates from the same batch of sonicated liposomes.

Having established a method for ultrasound-triggered enzyme activity, we next investigated whether we could use ultrasound to initiate a hydrogelation process. Specifically, we hypothesized that the calcium released by ultrasound-exposed liposomes could be used to trigger the transglutaminase-catalyzed hydrogelation of fibrinogen. Transglutaminase catalyzes intramolecular and intermolecular fibrinogen crosslinking, with the latter used to form fibrinogen hydrogels. We applied ultrasound for 3, 10 or 50 s (20 kHz frequency, 25% duty cycle, 20% amplitude) to a liquid solution of calcium-loaded liposomes, and monitored the transglutaminase-catalyzed hydrogelation of fibrinogen using time-resolved rheometry (1% strain, 1 rad s⁻ frequency). We observed a relatively rapid gelation in all cases, with the elastic modulus (G′) exceeding the viscous modulus (G″) within the first 30 min (FIG. 7d-f). Data shown is for one replicate. Measurements were performed at a frequency of 1 rad s⁻¹ and at 1% strain. The elastic modulus at the 5 h endpoint was dependent upon the initial ultrasound exposure time: 34, 55 and 177 Pa for 3, 5 and 10 s, respectively. Importantly, a rheology control experiment for liposomes with no ultrasound exposure revealed that the unexposed controls were liquid at 6 h, validating the role of ultrasound in the hydrogelation process (FIG. 8). Frequency (FIG. 8a) and strain sweeps (FIG. 8b) were performed on solutions of calcium-loaded liposomes, transglutaminase and fibrinogen that had not been exposed to ultrasound (measured after 6 h). This analysis showed these negative controls to be in liquid form, with the elastic modulus (G′, filled symbols) not exceeding the viscous modulus (G″, empty symbols). The frequency sweep was performed at 1% strain while the strain sweep was performed at 1 rad s⁻¹ frequency.

### Example 2: Ultrasound-Triggered Gelation of Fibrinogen Hydrogels Using Liposome-Microbubble Conjugates

### Microbubble Formulation and Sizing

Microbubbles were formulated using a method adapted from a previously reported protocol (Small 2014, 10, 3316). A lipid film comprising 1,2-distearoyl-sn-glycero-3-phosphocholine (DSPC), DSPE-PEG2000 and DSPE-PEG2000 biotin in an 86:9:5 molar ratio was hydrated with 0.6 M NaCl to a final lipid concentration of 6.32 mg mL⁻¹. The lipid suspension was vortexed for 15 s and heated at 75° C. for 2 min, then vortexed and heated once more. A perfluorohexane/air mixture was pumped over the lipid suspension and the sample was sonicated using a VibraCell probe sonicator (20 kHz, 40% amplitude, 100% duty cycle, 3 s). Four centrifuge washes (100 g, 3 min) were performed to remove excess lipid. Samples were imaged on an Olympus IX71 inverted microscope in bright field mode with a 60× oil immersion objective lens. Automate image analysis was performed using ImageJ. The average-shifted histogram was generated via the Buriak group data plotter website (https://maverick.chem.ualberta.ca/plot/ash).

### Microbubble-Liposome Conjugation

To form the conjugates, 400 μL biotinylated microbubbles were incubated with 21 μL of an aqueous 10 mg mL⁻¹ neutravidin solution for 15 min at 300 rpm and 22° C. in an Eppendorf Thermomixer Comfort. Four centrifuge washes were performed (100 g, 3 min) to remove any unbound neutravidin. 200 μL of neutravidin-functionalized microbubbles were then incubated with 200 μL of calcium-loaded liposomes for 30 min at 300 rpm and 22° C. in an Eppendorf Thermomixer Comfort. The mixture was prepared with 7×10⁵ liposomes per microbubble. Four centrifuge washes were performed (100 g, 3 min) to remove unbound liposomes. Microbubble-liposome conjugates were also imaged on a Leica SP5 inverted confocal microscope in bright field and fluorescence mode with a 63× oil immersion objective lens. For this experiment, microbubble-liposome conjugates were prepared using DiO-labelled liposomes and Dil-labelled microbubbles. 1,1′-dioctadecyl-3,3,3′,3′-tetramethylindocarbocyanine perchlorate (Dil) and 3,3′-dioctadecyloxacarbocyanine perchlorate (DiO) are lipid fluorescent dyes.

### Structured Illumination Microscopy (SIM)

Conjugates were prepared using DiO-labelled liposomes and unlabelled microbubbles, then diluted in glycerol to a concentration of 6×10⁶ conjugates mL⁻¹. 5 μL of this suspension was placed on a glass slide, covered with a coverslip and left to settle for 10 min before imaging. Micrographs were obtained on a Zeiss Elyra PS.1 microscope (Carl Zeiss) equipped with sCMOS PCO Edge using a Plan-Apochromat 63×1.4 NA oil-immersion DIC objective lens. Each image was recorded with three orientation angles of the excitation grid and five phases acquired for each image with a 110 nm z-step and a pixel size of 32 nm imaged at 8 bits per pixel with no image averaging. A 488 nm laser was used for imaging. SIM processing was performed using SIM module of the Zen software package (Carl Zeiss) while 3D SIM reconstruction was performed with Fiji ImageJ software (NIH).

### Ultrasound-Triggered Calcium Release from Microbubble-Liposome Conjugates

An o-CPC assay was used to quantify the total encapsulated calcium level of lysed liposome and microbubble-liposome conjugate suspensions. The remaining liposome and conjugate suspensions were then diluted to a total encapsulated calcium concentration of 100 μM. These dose-matched samples were then aliquoted, with 250 μL transferred into 500 μL DNA LoBind tubes. Ultrasound was applied for 5 s with a probe sonicator, before the quantity of released calcium was measured using a second o-CPC assay. Conjugates were also imaged with a camera and a bright field microscope (Olympus IX71) before and after ultrasound exposure.

### Ultrasound-Triggered Enzymatic Hydrogelation from Microbubble-Liposome Conjugates

125 μL of microbubble-liposome conjugates (total encapsulated calcium of 420 μM) were transferred into 500 μL DNA LoBind tubes. Ultrasound was applied for 5 s with a probe sonicator and a negative control was left without ultrasound exposure. 100 μL of each suspension was added to separate solutions of fibrinogen in 0.6 M NaCl (final fibrinogen concentration of 22.68 mg mL⁻¹) and aqueous DTT (final DTT concentration of 10 mM) in a 500 μL Protein LoBind tube. Transglutaminase was added to a final concentration of 5 μM and samples were incubated at 25° C. for 42 h. Frequency sweeps (0.1-10 rad s⁻¹ at 1% strain) and strain sweeps (0.1-100% at 1 rad s⁻¹) were performed after 42 h using an AR 2000 rheometer (TA Instruments) equipped with an 8 mm steel parallel plate and an oil chamber.

### Results and Discussion

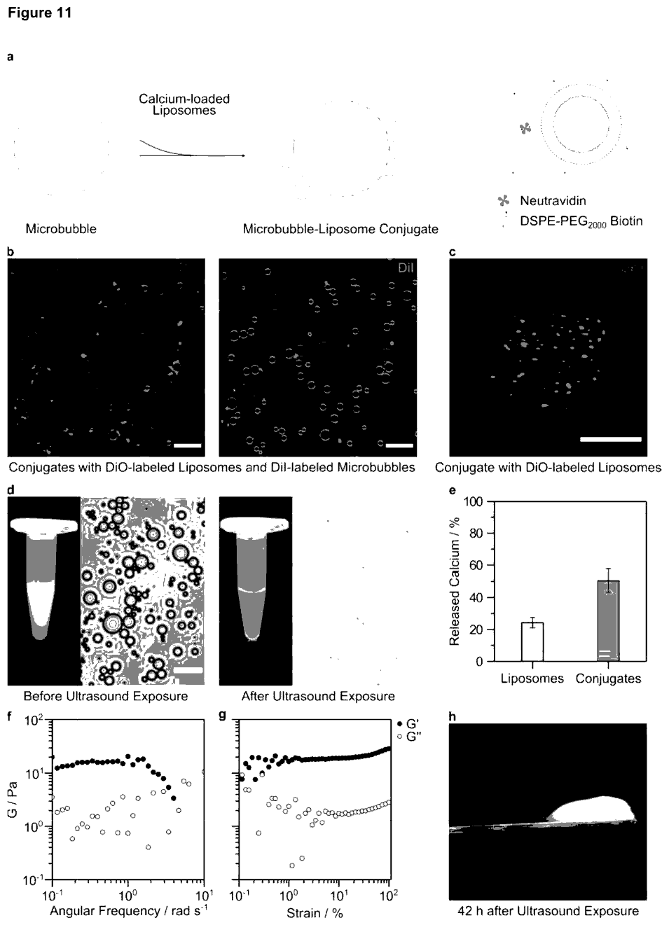

Having successfully demonstrated ultrasound-triggered enzyme catalysis and hydrogelation using calcium-loaded liposomes, we sought to extend our capabilities by integrating our technology with ultrasound-responsive gaseous microbubbles, which have been used extensively in drug delivery, ultrasound imaging, and thermal ablation. Conjugation of liposomes to microbubbles has previously been used to enhance the ultrasound-triggered release of liposomal cargo. Therefore, we investigated whether we could engineer microbubble-liposome conjugates capable of ultrasound-triggered fibrinogen hydrogelation. We produced biotinylated microbubbles by hydrating a lipid film comprising 1,2-distearoyl-sn-glycero-3-phosphocholine (DSPC), DSPE-PEG and DSPE-PEG₂₀₀₀ biotin in a molar ratio of 86:9:5, and then pumping the solution with a mixture of perfluorohexane and air. We used bright field microscopy to visualize the microbubbles and image analysis to measure a mean microbubble diameter of 2.5±1.6 μm (FIG. 10). We conjugated liposomes to the surface of the microbubbles by using neutravidin to bind with the biotin moieties present on both components. Using confocal fluorescence microscopy, we observed co-localization of fluorescently-labelled liposomes on the surface of fluorescently-labelled microbubbles, which indicated a successful conjugation. Further insight was provided by structured illumination microscopy, a super-resolution imaging technique that revealed liposomes uniformly distributed across the microbubble surface (FIG. 11). A schematic of the microbubble-liposome conjugation is provided in FIG. 11a. Confocal fluorescence microscopy showed fluorescently-labelled liposomes (shown in blue) colocalized with Dil-labelled microbubbles (shown in yellow), suggesting a successful conjugation (FIG. 11b). A z-projection, obtained using structure illumination microscopy, showed DiO-labelled liposomes (shown in blue) conjugated to a single microbubble obtained (FIG. 11c). Camera images and bright field microscopy showed intact microbubble-liposome conjugates before and after ultrasound exposure (20 kHz, 25% duty cycle, 20% amplitude, 5 s) (FIG. 11d). Scattering caused by intact conjugates gives the solution an opaque white appearance. The percentage of released calcium measured from dose-matched liposomes and liposome-microbubble conjugates after ultrasound exposure (20 kHz, 25% duty cycle, 20% amplitude, 5 s) is shown in FIG. 11e. Frequency (FIG. 11f) and strain (FIG. 11g) sweeps of the fibrinogen hydrogel obtained after 5 s ultrasound exposure and 42 h static gelation are provided. The strain sweep was performed at a frequency of 1 rad s⁻¹ while the frequency sweep was performed at 1% strain. A picture of a fibrinogen hydrogel, 42 h after the calcium-loaded liposome-microbubble conjugates were exposed to 5 s of ultrasound is provided in FIG. 11h.

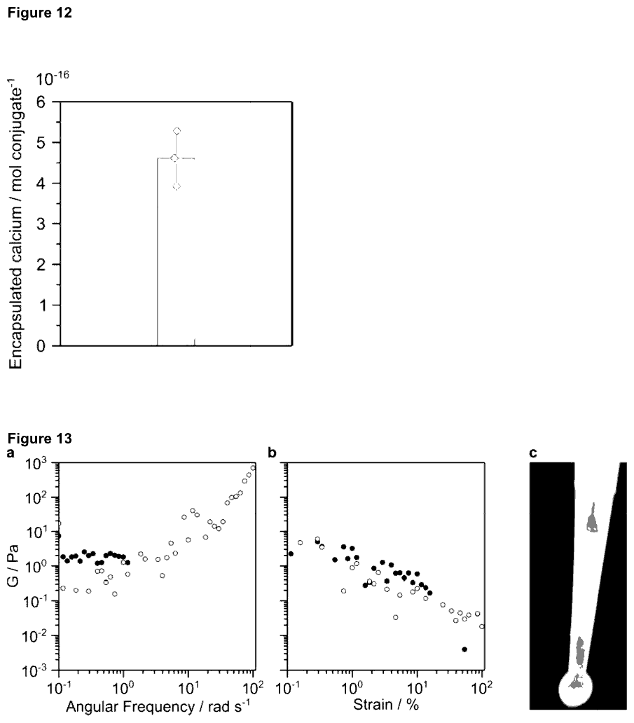

Using an o-CPC calcium assay, we measured approximately (4.6±0.6)×10⁻¹⁶ mol per conjugate (FIG. 12), a quantity that was sufficient to test ultrasound-triggered hydrogelation. Data shown are the mean and standard deviation of three independent batches.

We exposed the conjugates to ultrasound for 5 s, and then evaluated the suspension using bright field microscopy and an o-CPC calcium assay. We were unable to identify any microbubble-liposome conjugates after ultrasound exposure, indicating widespread destruction of the microbubble population. Under these conditions, the microbubble-liposome conjugates liberated approximately twice the amount of calcium (50±7%) than dose-matched liposomes (24±3%). This observation validated our hypothesis that microbubble conjugation would enhance the efficiency of liposomal calcium release. We next showed that by exposing calcium-loaded conjugates to 5 s of ultrasound, we could trigger transglutaminase-catalyzed hydrogelation of fibrinogen. At the 42-h endpoint, we measured a G′ of 21 Pa in the ultrasound-exposed system, with no gelation observed in the unexposed control group. This analysis showed these negative controls to be in liquid form, with the elastic modulus (G′, filled symbols) not exceeding the viscous modulus (G″, empty symbols). The frequency sweep was performed at 1% strain while the strain sweep was performed at 1 rad s⁻¹ frequency (FIG. 13).

For this demonstration, we used a relatively low level of total encapsulated calcium, which resulted in a longer hydrogelation process than for the liposome system, however, there is scope to increase hydrogelation kinetics by using a higher concentration of conjugates.

### Example 3: Ultrasound-Triggered Gelation of Alginate Hydrogels

Calcium-loaded liposomes were also shown to be able to induce alginate hydrogelation. Alginate is an anionic polysaccharide that can be crosslinked by divalent cations (e.g. Ca²⁺) and is widely used both in vitro cell studies and in human clinical trials.

The same liposomal formulation described in the ultrasound-triggered enzymatic gelation paper was used. Briefly, liposomes (DPPC:DSPE PEG biotin=99:1 mol ratio) were formulated via the interdigitation-fusion vesicle method. The total encapsulated calcium was measured with the o-cresolphthalein (o-CPC) assay following liposome lysis with Triton X-100 and was 32.4±0.8 mM.

### Temperature-Dependent Calcium Release

A temperature-dependent release experiment was conducted prior to the ultrasound exposure experiment. Here, 50 μL sample were put in 500 μL tubes immersed in a water bath set at the desired T. Samples were incubated for 15 minutes at each temperature (see Table 1) and a thermocouple was placed inside the test tube to monitor its temperature for the whole incubation time. At the end of the incubation time, samples were cooled down to 20° C.

TABLE 1
List of temperatures at which the liposomes were incubated.
Temperature ° C.

T₁ = 30
T₂ = 35
T₃ = 37
T₄ = 39
T₅ = 40
T₆ = 41
T₇ = 42
T₈ = 43
T₉ = 44
T₁₀ = 45
T₁₁ = 49

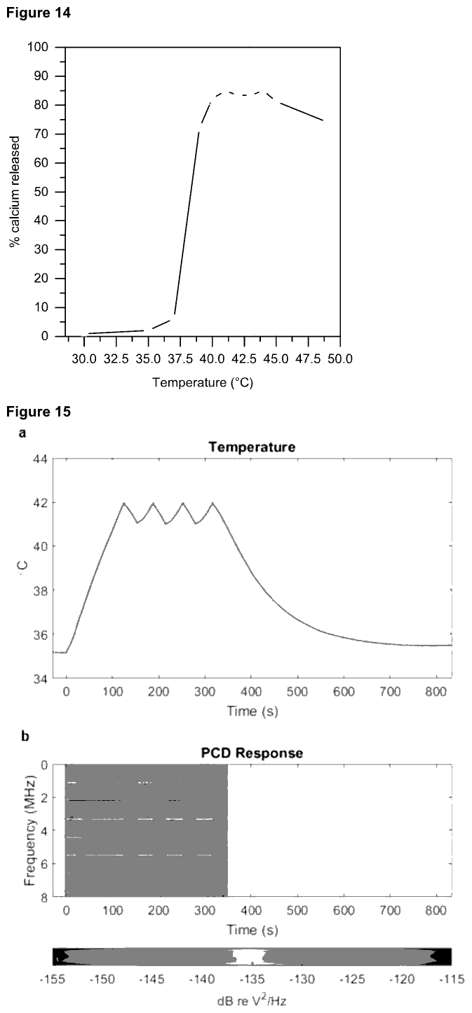

To measure the released calcium with the o-CPC assay, samples were diluted so to have a total encapsulated calcium concentration of 2 mM. A standard curve with no-calcium liposomes at matching particle concentration and spiked calcium was used in this case. The percentage of released calcium as a function of the temperature is reported in FIG. 14 (mean±s.d. for n=2 experimental replicates from the same batch of liposomes is reported). At 37° C., the released calcium is roughly 6% of the total but upon incubation of the liposomes at 39° C., roughly 73% of the total encapsulated calcium is released. Up to 85% of the total encapsulated calcium is released upon further increase of the temperature between 40 and 45° C.

### Ultrasound-Triggered Alginate Hydrogelation

To test the capability of high-frequency ultrasound to trigger alginate gelation, an apparatus comprising a source transducer with a focal diameter of 1.9 mm and a receiver for cavitation detection immersed in a water tank was used. The temperature of the water bath was held constant at 35° C. for the whole duration of the experiment.

Briefly, 525 μL of calcium loaded liposomes were mixed with 175 μL of 4 wt/vol % alginate solution in 0.6 M NaCl and loaded in the sample chamber. Ultrasound (1.1 MHz, 72% duty cycle, 65 mVₚₚ) was applied for 5 minutes and the sample temperature, which was monitored via a thermocouple, was kept between 41 and 42° C. (FIG. 15a). Furthermore, no cavitation was detected, as shown by the passive cavitation detection (PCD) map (FIG. 15b).

After the ultrasound exposure the samples were left to cool down and extracted from the sample holder. Gelation was achieved and the alginate hydrogels could be manually handled.

As a control, the calcium-loaded/alginate mixture was exposed to ultrasound (1.1 MHz, 72% duty cycle, 65 mVₚₚ, pulsed mode: 20 s ON, 20 s OFF) for approximately 14 min so to deliver the same total power while keeping the temperature between 37 and 38° C. Also in this case, temperature and cavitation were constantly monitored. No gelation was observed in this case, and the sample remained liquid, thus suggesting that this system may be suited for on-demand, ultrasound-triggered hydrogelation in vivo.

### Example 4: One-Pot Ultrasound-Triggered Fibrinogen Hydrogelation

The same protocol was followed as described in Example 1 (“Liposome Formulation”, “Ultrasound-Triggered Hydrogelation using Calcium-Loaded Liposomes”), with the following variations. Calcium-loaded liposomes, transglutaminase, and fibrinogen were mixed and exposed to ultrasound for 10 s. 100 μL of calcium-loaded liposomes were mixed with 1 μL DTT in deionized water (final DTT concentration of 8.69 mM) and 24.4 μL fibrinogen in 0.6 M NaCl (final fibrinogen concentration of 22.42 mg mL⁻¹). Ultrasound was applied to the mixture (20 kHz, 25% duty cycle, 20% amplitude) and time-sweep rheometry using 1% strain and 1 rad s⁻¹ was performed at 25° C. with an AR 2000 rheometer (TA instruments) equipped with an 8 mm steel parallel plate and an oil chamber to prevent solvent evaporation.

### Results and Discussion

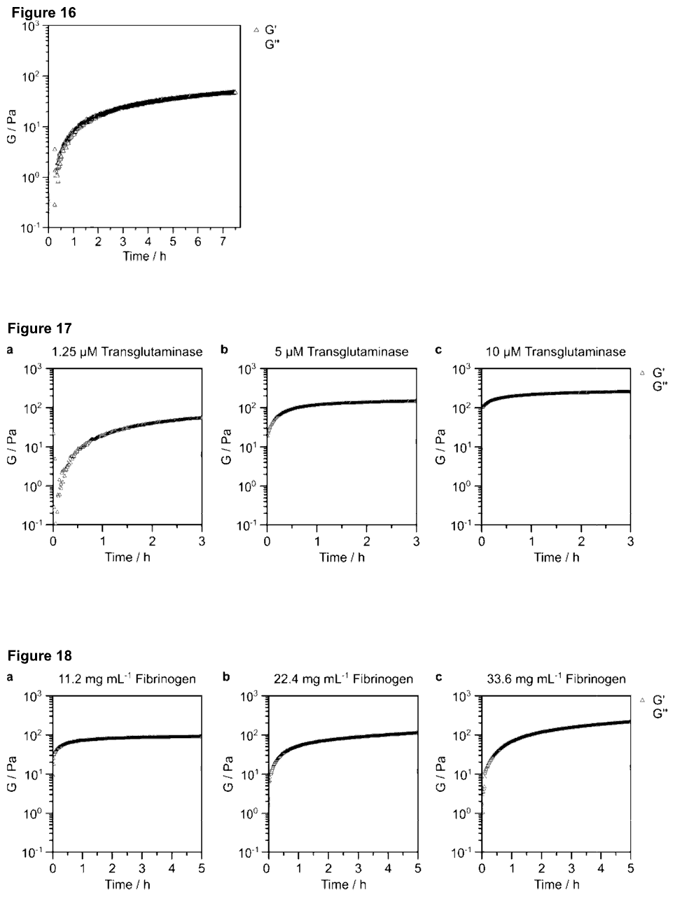

A mixture of calcium-loaded liposomes, fibrinogen (final concentration of 22.4 mg mL⁻¹), and transglutaminase (final concentration of 5 μM) was sonicated for 10 s. Time-sweep rheometry using 1% strain and 1 rad s⁻¹ was performed at 25° C. (approximately 10 minutes after the ultrasound stimulation). Gelation occurred relatively quickly, as shown in FIG. 16 by the elastic modulus (G′) exceeding the viscous modulus (G″). This example therefore demonstrates that ultrasound can effectively trigger enzyme-catalyzed hydrogelation when all components are present during ultrasound exposure.

### Example 5: Ultrasound-Triggered Hydrogelation With Varying Transglutaminase Concentration

The same protocol was followed as described in Example 1 (“Liposome Formulation”, “Ultrasound-Triggered Hydrogelation using Calcium-Loaded Liposomes”), with the following variations. Calcium-loaded liposomes were exposed to ultrasound for 50 s. Transglutaminase was added to a final concentration of 1.25, 5 or 10 μM immediately prior to rheological measurements. A time sweep was performed over 3 h at 1% strain and 1 rad s⁻¹ with an AR 2000 rheometer (TA instruments) equipped with an 8 mm steel parallel plate and an oil chamber to prevent solvent evaporation.

### Results and Discussion

The gelation of fibrinogen was measured using time-sweep rheometry upon the addition of (a) 1.25 μM, (b) 5 μM and (c) 10 μM transglutaminase (FIG. 17). An increase in the gelation kinetics was observed with increasing transglutaminase concentration. Moreover, we demonstrated that gelation kinetics can be increased by doubling the transglutaminase concentration from 5 μM to 10 μM (FIG. 17). Here, the gelation occurred so fast that the first datapoints measured on the rheometer were well beyond the linear region, with G′ already exceeding 90 Pa at the first datapoint measured. We also showed that the gelation could be slowed by reducing the transglutaminase concentration to 1.25 μM.

This example shows that faster or slower hydrogelation can be achieved simply by increasing or decreasing the transglutaminase concentration, respectively; thus, allowing tuning of gelation rate.

### Example 6: Ultrasound-Triggered Hydrogelation With Varying Fibrinogen Concentration

The same protocol was followed as described in Example 1 (“Liposome Formulation”, “Ultrasound-Triggered Hydrogelation using Calcium-Loaded Liposomes”), with the following variations. Calcium-loaded liposomes were exposed to ultrasound for 50 s. Fibrinogen was added to a final concentration of 11.2 mg mL⁻¹, 22.4 mg mL⁻¹ or 33.6 mg mL⁻¹ immediately prior to rheological measurements. A time sweep was performed over 5 h at 1% strain and 1 rad s−1with an AR 2000 rheometer (TA instruments) equipped with an 8 mm steel parallel plate and an oil chamber to prevent solvent evaporation. Where fibrinogen was added to a final concentration of 33.6 mg mL⁻¹, a time sweep was also performed over 23 h.

### Results and Discussion

The transglutaminase-catalyzed fibrinogen gelation upon ultrasound exposure was measured using time sweep rheology after the application of ultrasound for 50 s to calcium-loaded liposomes. After 5 h, the elastic moduli were measured as 90, 110 and 211 Pa for (a) 11.2 mg mL⁻¹, (b) 22.4 mg mL⁻¹ and (c) 33.6 mg mL⁻¹ fibrinogen, respectively (FIG. 18).

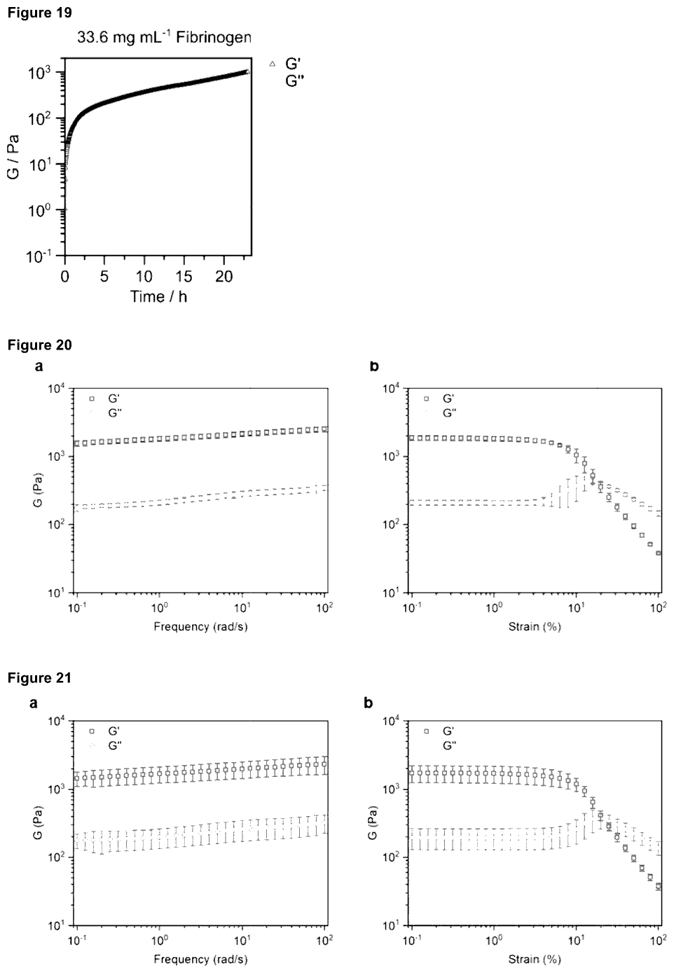

Ultrasound-triggered hydrogelation with increased crosslinking time was carried out using a 33.6 mg mL⁻¹ fibrinogen solution (FIG. 19). The transglutaminase-catalyzed fibrinogen gelation upon ultrasound exposure was measured using time sweep rheology after the application of ultrasound for 50 s to calcium-loaded liposomes. After 23 h, the elastic modulus was 1009 Pa. The elastic modulus (G′) was increased by raising the initial concentration of fibrinogen to 33.6 mg mL⁻¹ (FIG. 18). Moreover, at this concentration, the gel was still increasing in elastic modulus after 5 h, and exceeded 1 kPa after 23 h of measurement (FIG. 19).

This example shows that the elastic modulus can be tuned by changing the fibrinogen concentration or by increasing the crosslinking time, thus allowing tuning of the hydrogel mechanical properties.

### Example 7: Ultrasound-Triggered Gelation of Alginate Hydrogels in the Presence of Glass Microspheres

### Materials

1,2-dipalmitoyl-sn-glycero-3-phosphocholine (DPPC) and 1,2-distearoyl-sn-glycero-3-phosphoethanolamine-N4-[methoxy(polyethylene glycol)-2000] (ammonium salt) (DSPE-PEG2000) were purchased from Sigma Aldrich and produced by Avanti Polar Lipids. All other reagents were purchased from Sigma Aldrich. Ultrapure water (18.2 MΩ cm) was taken from TR Duo10 UF Polisher triple (Triple Red, Avidity Science). Glass microspheres (Soda Lime Solid Glass Microspheres 2.5 g/cc 5-50 um) were bought from Cospheric.

### Liposome Formulation

Calcium-loaded liposomes were formulated using an established interdigitation-fusion vesicle method (Biochim. Biophys. Acta—Biomembr. 1994, 1195, 237). Briefly, a solution of 99 mol % of DPPC and 1 mol % of DSPE-PEG2000 was prepared in chloroform, dried with a stream of nitrogen gas in a glass vial and then kept under vacuum for at least 3 h. The lipid film was hydrated to a lipid concentration of 20 mg mL⁻¹ with an aqueous solution containing 0.2 M CaCl₂ for 1 h at 55° C. under constant stirring. The liposome solution was extruded 25 times through a 100 nm polycarbonate membrane and 31 times through a 50 nm polycarbonate membrane (Whatman® Nucleopore Track-Etched™ membranes) at 55° C. To induce interdigitation, ethanol was added to a final concentration of 4 M while stirring. The interdigitated gels were stored overnight at 4° C. Five centrifuge washes (first wash at 8500 g for 8 min, second wash at 8000 g for 8 min, remaining washes at 8000 g for 6 min) were performed to remove the ethanol, after which the lipid gels were incubated at 65° C. for 2.5 h (650 rpm) to form large unilamellar liposomes. These liposomes were then extruded 31 times through a 400 nm polycarbonate membrane (Whatman® Nucleopore Track-Etched™ membranes) at 55° C. to yield a monodisperse population of unilamellar vesicles. The calcium-loaded liposomes were dialyzed against iso-osmotic buffer (0.3 M NaCl) to remove free calcium, and then stored at 4° C. prior to use.

### Ultrasound-Triggered Alginate Hydrogelation

To test the capability of high-frequency ultrasound to trigger alginate gelation, an apparatus comprising a focused transducer and a confocal receiver for cavitation detection immersed in a water tank was used. The temperature of the water bath was held constant at 35° C. for the whole duration of the experiment. Briefly, 500 μL of calcium loaded liposomes were mixed with 500 μL of 4 wt/vol % alginate solution in MilliQ water containing 60 mM 2-[4-(2-hydroxyethyl)piperazin-1-yl]ethanesulfonic acid (HEPES), and 150 mg glass microspheres and loaded in the sample chamber (resulting in a mixture containing 2 wt/v % alginate and 6 v/v % glass microspheres). Prior to ultrasound application, the mixture was degassed 3 times for 3 min in a vacuum chamber. Ultrasound (1.1 MHz, 75% duty cycle, 1.3 MPa peak pressure, 1.9 mm focal diameter) was applied so that the sample temperature, which was monitored via a thermocouple, was kept between 39.5 and 40.5° C. for 60 seconds. Alternatively, higher frequency ultrasound (3.3 MHz, 75% duty cycle, 3.8 MPa peak pressure, 0.63 mm focal diameter) was used. After the ultrasound exposure the samples were left to cool down and extracted from the sample holder. Gelation was achieved and the alginate hydrogels could be manually handled.

### Rheology of Ultrasound-Triggered Hydrogels

The mechanical properties of the obtained hydrogels were characterized by rheometry. An AntonPaar MCR 302 rheometer equipped with a 25 mm steel parallel plate and a water trap to prevent solvent evaporation. The samples were loaded on the rheometer plate and the 25 mm steel parallel plate (upper plate) was lowered to have a gap of 0.3 mm. The frequency sweep (0.1-100 rad s⁻¹) was performed at 0.5% strain while the strain sweep (0.01-100%) was performed at 1 rad s⁻¹. The output values from the rheometer are the elastic and viscous moduli G′ and G″.

### Results and Discussion

These results demonstrate that it is possible to achieve alginate gelation by exposing a mixture of alginate, glass microspheres, and calcium-loaded liposomes to ultrasound operated at 1.1 MHz (70 mVₚₚ, 75% duty cycle, 40 s exposure) or 3.3 MHz (126 mVₚₚ, 75% duty cycle, 60-80 s exposure). The strain and frequency sweeps of the obtained hydrogels are shown in FIG. 20 (1.1 MHz) and FIG. 21 (3.3 MHz). The data shown in these figures is mean and standard deviation of 3 replicates.

The utility of using low-MHz frequency ultrasound (e.g. 1.1 MHz or 3.3 MHz) is that it allows noninvasive triggering for in vivo applications using well-established ultrasound devices and physics. Increasing the frequency results in a decrease in wavelength and an improvement in the spatial precision of the triggering for a fixed ultrasound source size. Beneficially, the intrinsic ability to convert ultrasound energy into heat (ultrasonic absorption) increases with frequency.

Glass microspheres were used to further enhance the absorption of the alginate mixture. For in vivo use, increasing the ultrasonic absorption of the formulation so that it is at least as high as the surrounding tissue results in heat being generated preferentially at the intended gelation site. In principle, this gives the most controlled and efficient triggering process.

The present invention provides a new approach to achieve ultrasound-triggered enzyme catalysis, as demonstrated by ultrasound-triggered enzymatic hydrogelation. We have shown that a brief exposure to ultrasound (1-50 secs) could be used to controllably liberate liposomal calcium, which could subsequently activate transglutaminase catalysis. We used this ultrasound-triggered catalysis to enzymatically crosslink fibrinogen and form self-supporting, viscoelastic hydrogels. This was also demonstrated with alginate. Importantly, the calcium release, enzyme kinetics and gelation rate can all be tuned by varying the ultrasound exposure time. We also demonstrated that calcium-loaded liposomes could be conjugated to gaseous microbubbles to enhance the payload release upon ultrasound exposure. These calcium-loaded microbubble-liposome conjugates were also used for ultrasound-activated hydrogelation of fibrinogen. We also demonstrated that gelation rate may be tuned by varying the concentration of transglutaminase and that the hydrogel mechanical properties can be tuned by changing the fibrinogen concentration or by increasing the crosslinking time. Taken together, these results represent an entirely new class of stimuli for enzyme activity and hydrogelation that sit alongside the traditional triggers of light, pH, temperature and chemical addition. While transglutaminase was used as an exemplar in this work, the same principles could be applied to other enzymes with ionic cofactors, which include many oxidoreductases, transferases, hydrolases, lyases, isomerases and ligases.

The versatility of this technique extends beyond fibrinogen and alginate hydrogelation, opening up a wide range of opportunities for ultrasound-triggered molecular biology, synthetic biology and material science.

Those of skill in the art will recognize that the invention can be practiced in a variety of embodiments and that the foregoing description and examples are for purposes of illustration and not limitation of the claims that follow. It will be appreciated that variations of the described embodiments may be made which are still within the scope of the invention. Changes that come within the meaning and range of equivalency of the claims are intended to be embraced therein.

1. A process for gelation, wherein the process comprises the steps of:
a) providing a mixture comprising a liposome and a gel precursor; wherein the liposome encapsulates a payload that is capable of inducing gelation of the gel precursor;
b) applying ultrasound to the mixture; to trigger release of the payload from the liposome and induce gelation of the gel precursor.

2. The process of claim 1, wherein the payload acts directly on the precursor to induce gelation or the payload acts indirectly on the precursor to induce gelation (e.g. by activation of an enzyme).

3. The process of claim 1, wherein the mixture further comprises a cofactor-dependent enzyme in its inactive form; and
the payload is a cofactor that is capable of activating the enzyme;
wherein applying ultrasound to the mixture triggers release of the cofactor from the liposome, which activates the enzyme.

4. The process of claim 3, wherein the cofactor is an ionic cofactor.

5. The process of claim 3, wherein the enzyme is a transglutaminase, oxidoreductase, peroxidase, transferase, alcohol dehydrogenase, hydrolase, lyase, isomerase or ligase, or a combination thereof; optionally wherein the enzyme is transglutaminase.

6. A process for ultrasound-triggered enzyme catalysis, wherein the process comprises the steps of:
a) providing a mixture comprising a liposome, a cofactor-dependent enzyme in its inactive form, and a substrate of the enzyme; wherein the liposome encapsulates a cofactor that is capable of activating the enzyme;
b) applying ultrasound to the mixture; to trigger release of the cofactor from the liposome and activate the enzyme.

7. The process of claim 6, wherein the cofactor is an ionic cofactor.

8. The process of claim 6, wherein the enzyme is a transglutaminase, oxidoreductase, peroxidase, transferase, hydrolase, alcohol dehydrogenase, lyase, isomerase or ligase, or a combination thereof; optionally wherein the enzyme is transglutaminase.

9. The process of claim 6, wherein the substrate is a gel precursor and the activated enzyme induces gelation of the gel precursor.

10. The process of claim 1, wherein the gelation is hydrogelation and the gel precursor is a hydrogel precursor.

11. The process of claim 1, wherein the substrate, gel precursor, or hydrogel precursor is selected from fibrinogen, collagen, alginate, poly(ethylene glycol), poly(vinyl alcohol), poly(acrylic acid), or a methacrylate-, tetrazine-, or norbornene-modified biopolymer; optionally wherein the substrate, gel precursor, or hydrogel precursor is fibrinogen.

12. The process of claim 1, wherein the payload or cofactor is a metal ion.

13. The process of claim 12, wherein the metal ion is a calcium, zinc, iron, magnesium, aluminium, barium or strontium ion, or a combination thereof; optionally wherein the metal ion is a calcium ion).

14. The process of claim 3, wherein:
a) the cofactor is a zinc ion and the enzyme is an alcohol dehydrogenase, lyase, or hydrolase; or
b) the cofactor is a calcium ion and the enzyme is phospholipase A, acyltransferase, or transglutaminase; or
c) the cofactor is an iron ion and the enzyme is an alkaline phosphatase; or
d) the cofactor is a calcium ion, the enzyme is transglutaminase and the gel precursor is fibrinogen; or
e) the cofactor is a calcium ion, the enzyme is transglutaminase and the gel precursor comprises poly(ethylene glycol) (PEG) and hyaluronic acid (HA); or
f) the cofactor is a calcium ion, the enzyme is peroxidase, and the gel precursor comprises tyramine and hyaluronic acid; or
g) the cofactor is a calcium ion, the enzyme is phospholipase A and the gel precursor is a phospholipid; or
h) the cofactor is a calcium ion, the enzyme is an acyltransferase and the gel precursor is a molecule containing an acyl moiety; or
i) the cofactor is a zinc ion, the enzyme is an alcohol dehydrogenase and the gel precursor is an alcohol; or
j) the cofactor is an iron ion, the enzyme is an alkaline phosphatase and the gel precursor is a molecule containing a phosphate moiety.

15. A process for the release of a payload from a liposome, wherein the process comprises the step of applying ultrasound to a liposome encapsulating a payload; and the payload is a metal ion (e.g. a calcium ion).

16. (canceled)

17. The process of claim 1, wherein the mixture further comprises a liquid vehicle.

18. The process of claim 1, wherein the liposome is conjugated to a microbubble.

19. The process of claim 1, wherein the liposome comprises one or more phosphatidylcholine; optionally wherein the liposome comprises DPPC and DSPE-PEG₂₀₀₀ biotin.

20. The process of claim 3, wherein:
the liposome comprises DPPC and DSPE-PEG2000 biotin;
the cofactor is a calcium ion;
the enzyme is transglutaminase; and
the gel precursor is fibrinogen.

21. The process of claim 1, wherein:
the liposome comprises DPPC and DSPE-PEG₂₀₀₀ biotin;
the payload is a calcium ion; and
the gel precursor is alginate.

22. The process of claim 1, wherein:
a) ultrasound is applied for at least about 1 second and/or at a frequency of at least about 20 kHz; and/or
b) ultrasound is focused to a region of at least about 0.5 mm³.

23-25. (canceled)
